SEQUENCE LISTING (37 sequences)

SEQ ID NO: 1 - protein, 76 aa, Homo sapiens [sequence omitted]

SEQ ID NO: 2 - protein, 76 aa, Artificial
  ubiquitin mutant
CQIFVKTLTGKTITLEVEPSDTIENVKAKIQDKEGIPPDQQRLIFAGKQLEDGRTLSDYNIQKESTLHLVLRLRGG

SEQ ID NO: 3 - protein, 85 aa, Artificial
  ubiquitin mutant
YPYDVPDYAMQIFVKTLTGKTITLEVEPSDTIENVKAKIQDKEGIPPDQQRLIFAGKQLEDGRTLSDYNIQKESTLHLVLRLRGG

SEQ ID NO: 4 - protein, 82 aa, Artificial
  ubiquitin mutant
HHHHHHMQIFVKTLTGKTITLEVEPSDTIENVKAKIQDKEGIPPDQQRLIFAGKQLEDGRTLSDYNIQKESTLHLVLRLRGG

SEQ ID NO: 5 - protein, 84 aa, Artificial
  ubiquitin mutant
RRRRRRRRMQIFVKTLTGKTITLEVEPSDTIENVKAKIQDKEGIPPDQQRLIFAGKQLEDGRTLSDYNIQKESTLHLVLRLRGG

SEQ ID NO: 6 - protein, 86 aa, Artificial
  ubiquitin mutant
RQIKWFQNRRMQIFVKTLTGKTITLEVEPSDTIENVKAKIQDKEGIPPDQQRLIFAGKQLEDGRTLSDYNIQKESTLHLVLRLRGG

SEQ ID NO: 7 - protein, 87 aa, Artificial
  ubiquitin mutant
YGRKKRRQRRRMQIFVKTLTGKTITLEVEPSDTIENVKAKIQDKEGIPPDQQRLIFAGKQLEDGRTLSDYNIQKESTLHLVLRLRGG

SEQ ID NO: 8 - protein, 76 aa, Artificial
  ubiquitin mutant
XQIFVKTLTGKTITLEVEPSDTIENVKAKIQDKEGIPPDQQRLIFAGKQLEDGRTLSDYNIQKESTLHLVLRLRGG

SEQ ID NO: 9 - protein, 76 aa, Artificial
  ubiquitin mutant
MQIFVXTLTGKTITLEVEPSDTIENVKAKIQDKEGIPPDQQRLIFAGKQLEDGRTLSDYNIQKESTLHLVLRLRGG

SEQ ID NO: 10 - protein, 76 aa, Artificial
  ubiquitin mutant
MQIFVKTLTGXTITLEVEPSDTIENVKAKIQDKEGIPPDQQRLIFAGKQLEDGRTLSDYNIQKESTLHLVLRLRGG

SEQ ID NO: 11 - protein, 76 aa, Artificial
  ubiquitin mutant
MQIFVKTLTGKTITLEVEPSDTIENVXAKIQDKEGIPPDQQRLIFAGKQLEDGRTLSDYNIQKESTLHLVLRLRGG

SEQ ID NO: 12 - protein, 76 aa, Artificial
  ubiquitin mutant
MQIFVKTLTGKTITLEVEPSDTIENVKAXIQDKEGIPPDQQRLIFAGKQLEDGRTLSDYNIQKESTLHLVLRLRGG

SEQ ID NO: 13 - protein, 76 aa, Artificial
  ubiquitin mutant
MQIFVKTLTGKTITLEVEPSDTIENVKAKIQDXEGIPPDQQRLIFAGKQLEDGRTLSDYNIQKESTLHLVLRLRGG

SEQ ID NO: 14 - protein, 76 aa, Artificial
  ubiquitin mutant
MQIFVKTLTGKTITLEVEPSDTIENVKAKIQDKEGIPPDQQRLIFAGXQLEDGRTLSDYNIQKESTLHLVLRLRGG

SEQ ID NO: 15 - protein, 76 aa, Artificial
  ubiquitin mutant
MQIFVKTLTGKTITLEVEPSDTIENVKAKIQDKEGIPPDQQRLIFAGKQLEDGRTLSDYNIQXESTLHLVLRLRGG

SEQ ID NO: 16 - protein, 76 aa, Artificial [sequence omitted]

SEQ ID NO: 17 - protein, 76 aa, Artificial [sequence omitted]

SEQ ID NO: 18 - protein, 76 aa, Artificial [sequence omitted]

SEQ ID NO: 19 - protein, 76 aa, Artificial [sequence omitted]

SEQ ID NO: 20 - protein, 76 aa, Artificial [sequence omitted]

SEQ ID NO: 21 - protein, 76 aa, Artificial [sequence omitted]

SEQ ID NO: 22 - protein, 76 aa, Artificial [sequence omitted]

SEQ ID NO: 23 - protein, 76 aa, Artificial [sequence omitted]

SEQ ID NO: 24 - protein, 76 aa, Artificial [sequence omitted]

SEQ ID NO: 25 - protein, 76 aa, Artificial [sequence omitted]

SEQ ID NO: 26 - protein, 76 aa, Artificial [sequence omitted]

SEQ ID NO: 27 - protein, 5 aa, Artificial
  artificial peptide
LLFLV

SEQ ID NO: 28 - protein, 6 aa, Artificial
  artificial peptide
YGGFLX

SEQ ID NO: 29 - protein, 5 aa, Artificial
  artificial peptide
YGXFL

SEQ ID NO: 30 - DNA, 27 bp, Artificial
  primer
cttggtaacacttcttacatgaatgcc

SEQ ID NO: 31 - DNA, 27 bp, Artificial
  primer
ggcattcatgtaagaagtgttaccaag

SEQ ID NO: 32 - DNA, 26 bp, Artificial
  primer
cctgacggcaactctttctatcgggc

SEQ ID NO: 33 - DNA, 26 bp, Artificial
  primer
gcccgatagaaagagttgccgtcagg

SEQ ID NO: 34 - DNA, 26 bp, Artificial
  primer
gggatgggaactccttctacagggcc

SEQ ID NO: 35 - DNA, 26 bp, Artificial
  primer
ggccctgtagaaggagttcccatccc

SEQ ID NO: 36 - protein, 7 aa, Artificial
  SUMO peptide
YQEQTGX

SEQ ID NO: 37 - protein, 7 aa, Artificial
  SUMO peptide
FQQQTGX

(19) United States
(12) Patent Application Publication
(10) Pub. No.: US 2015/0158931 A1
(43) Pub. Date: Jun. 11, 2015

(54) CYSTEINE PROTEASE CAPTURING AGENTS

(71) Applicant: Stichting Het Nederlands Kanker Instituut, Amsterdam, (NL)

(72) Inventors: Huib Ovaa, Amsterdam (NL); Reggy Ekkebus, Amsterdam (NL); Sander Izaäk Van Kasteren, Amsterdam (NL); Annemieke De Jong, Amsterdam (NL); Paulus Petrus Geurink, Amsterdam (NL)

(73) Assignee: Stichting Het Nederlands Kanker Instituut, Amsterdam, (NL)

(21) Appl. No.: 14/412,990

(22) PCT Filed: Jul. 5, 2013
(86) PCT No.: PCT/NL2013/050501

§ 371 Date: Jan. 5, 2015

Related U.S. Application Data
(60) Provisional application No. 61/668,513, filed on Jul. 6, 2012.

(30) Foreign Application Priority Data
Jul. 6, 2012 (NL) 2009144
Nov. 28, 2012 (NL) 2009892

(51) Int. Cl. C07K 14/81; A61K 38/08; C07K 1/107; C07K 7/06; A61K 49/00; A61K 38/17

(52) U.S. Cl. CPC C07K 14/81; A61K 49/0004; A61K 38/1709; C07K 1/1077; C07K 7/06; A61K 38/08

(57) ABSTRACT

The invention concerns cysteine protease capturing agents capable of highly selective and irreversible binding of the corresponding cysteine protease. Such compounds may have utility in fundamental biological research and diagnostics, e.g. involving labeled or immobilized versions of such compounds, and they may also have potential utility in therapy, based on competitive inhibition of the cysteine protease, as will be readily apparent to those skilled in the art. The present inventors have discovered that such capturing agents can be obtained by modification of a cleavage fragment of a ‘natural’ substrate for the cysteine protease of interest, said modification involving the introduction of a propargyl moiety in such a way that the terminal alkyne group is positioned to allow for interaction with the free thiol group of the cysteine residue at the active site of the protease.

### FIELD OF THE INVENTION

The invention concerns cysteine protease capturing agents, their production and their various uses. In particular the invention concerns modified cleavage fragments of cysteine protease substrates capable of highly selective and irreversible binding of the corresponding cysteine protease.

### BACKGROUND OF THE INVENTION

Cysteine proteases are a class of proteases having as a common feature a catalytic mechanism involving nucleophilic cysteine thiol in the enzyme's active cite by an adjacent amino acid with a basic side chain, usually a histidine residue. Cysteine proteases play multi-faceted roles, virtually in every aspect of physiology and development. In humans they are responsible for apoptosis, MHC class II immune responses, pro-hormone processing, and extracellular matrix remodeling important to bone development. The ability of macrophages and other cells to mobilize elastolytic cysteine proteases to their surfaces under specialized conditions may also lead to accelerated collagen and elastin degradation at sites of inflammation in diseases such as atherosclerosis and emphysema.

Among the family of cysteine proteases are deubiquitinating proteases, cathepsins, SUMO proteases, calpains and caspases.

Deubiquitinating enzymes (DUBs) regulate ubiquitin-dependent metabolic pathways by cleaving ubiquitin-protein bonds. DUBs are also commonly referred to as deubiquitinating peptidases, deubiquitinating isopeptidases, deubiquitinases, ubiquitin proteases, ubiquitin hydrolyases, ubiquitin isopeptidases, or DUbs. The human genome encodes nearly 100 DUBs with specificity for ubiquitin in five gene families. DUBs play several roles in the ubiquitin pathway. First, DUBs carry out activation of the ubiquitin proproteins, probably cotranslationally. Second, DUBs recycle ubiquitin that may have been accidentally trapped by the reaction of small cellular nucleophiles with the thiol ester intermediates involved in the ubiquitination of proteins. Third, DUBs reverse the ubiquitination or ubiquitin-like modification of target proteins. Fourth, DUBs are also responsible for the regeneration of monoubiquitin from unanchored polyubiquitin, i.e., free polyubiquitin that is synthesized de novo by the conjugating machinery or that has been released from target proteins by other DUBs. Finally, the deubiquitinating enzymes UCH-L3 and YUH1 are able to hydrolyse mutant ubiquitin UBB+1 despite of the fact that the glycine at position 76 is mutated. Potentially, DUBs may act as negative and positive regulators of the ubiquitin system. In addition to ubiquitin recycling, they are involved in processing of ubiquitin precursors, in proofreading of protein ubiquitination and in disassembly of inhibitory ubiquitin chains. Deubiquitinating enzymes may be associated with disease.

Small Ubiquitin-like Modifier (or SUMO) proteins are a family of small proteins that are covalently attached to and detached from other proteins in cells to modify their function. SUMOylation is a post-translational modification involved in various cellular processes, such as nuclear-cytosolic transport, transcriptional regulation, apoptosis, protein stability, response to stress, and progression through the cell cycle. SUMO proteins are similar to ubiquitin, and SUMOylation is directed by an enzymatic cascade analogous to that involved in ubiquitination. SUMO proteases

Cathepsins are found in many types of cells including those in all animals. There are approximately a dozen members of this family, which are distinguished by their structure, catalytic mechanism, and which proteins they cleave. To date, a number of cathepsin have been identified and sequenced from a number of sources; for example, cathepsin B, F, H, L, K, S, W, and Z have been cloned. Most of the members become activated at the low pH found in lysosomes. Thus, the activity of this family lies almost entirely within those organelles. Many cathepsins belong to the papain superfamily of cysteine proteases. These proteases function in the normal physiological as well as pathological degradation of connective tissue. Cathepsins play a major role in intracellular protein degradation and turnover and remodeling. Cathepsin L is implicated in normal lysosomal proteolysis as well as several diseases states, including, but not limited to, metastasis of melanomas. Cathepsin S is implicated in Alzheimer's disease and certain autoimmune disorders, including, but not limited to juvenile onset diabetes, multiple sclerosis, pemphigus vulgaris, Graves' disease, myasthenia gravis, systemic lupus erythemotasus, rheumatoid arthritis and Hashimoto's thyroiditis; allergic disorders, including, but not limited to asthma; and allogenic immunbe responses, including, but not limited to, rejection of organ transplants or tissue grafts. Increased Cathepsin B levels and redistribution of the enzyme are found in tumors, suggesting a role in tumor invasion and metastasis. In addition, aberrant Cathepsin B activity is implicated in such disease states as rheumatoid arthritis, osteoarthritis, pneumocystisis carinii, acute pancreatitis, inflammatory airway disease and bone and joint disorders.

The calpain family of proteolytic enzymes is comprised of ubiquitous and tissue-specific isoforms of Ca²⁺-activated cysteine proteases that modify the properties of substrate proteins by cleavage at a limited number of specific sites generating large, often catalytically active fragments. The regulatory function of calpains is in contrast to the digestive functions of, for instance, the lysosomal proteases or the proteasome. Proteolysis by calpains is involved in a wide range of cellular functions, including cellular differentiation, integrin-mediated cell migration, cytoskeletal remodeling and apoptosis. Calpains have also been implicated in a number of neurodegenerative diseases, including brain injury, Alzheimer's disease, Parkinson's disease and Huntington's disease.

Caspases comprise a family of cysteine protease enzymes with a well-known role as key mediators in apoptosis signaling pathways and cell disassembly. Interleukin converting enzyme (ICE), also known as Caspase-1, was the first identified caspase. In humans, 11 other known caspases have been further identified. Caspases have been classified in two general groups according to their effects: proapoptotic (caspase-2, 3, 6, 7, 8, 9, 10) and proinflammatory (caspase-1, 4, 5, 11, 12) caspases. The proapoptotic caspases have been divided in initiators (caspase-2, 8, 9, 10) also known as group II, and executioners (caspase-3,6,7) of the apoptotic process or group III. The Interleukin converting enzyme (ICE) also known as Caspase-1 has a proinflammatory role only. There is growing evidence demonstrating the role of caspases in very diverse pathologies, such as cardiovascular disorders, tumor progression, response to pathogenic infection as well as in inflammatory and autoimmune disorders, neurodegenerative diseases and trauma.

As will be understood from the above, agents capable of selectively capturing cysteine proteases would have a wide variety of potential applications. It is therefore an object of the present invention to provide compounds that capture cysteine proteases, especially those described here above, in an irreversible and highly selective manner. Such compounds may have utility in fundamental biological research and diagnostics, e.g. involving labeled or immobilized versions of such compounds, and they may also have potential utility in therapy, based on competitive inhibition of the cysteine protease, as will be readily apparent to those skilled in the art.

### SUMMARY OF THE INVENTION

The present inventors have discovered that this objective can be accomplished by modification of a cleavage fragment of a ‘natural’ substrate for the cysteine protease of interest, said modification involving the introduction of a propargyl moiety in such a way that the terminal alkyne group is positioned to allow for interaction with the free thiol group of the cysteine residue at the active site of the protease.

The propargyl and thiol groups have proven to be sufficiently reactive towards each other, resulting in the formation of covalent bonds. This finding was highly surprising as the propargyl moiety is commonly used as a reagent in so-called ‘click chemistry’ or bioorthogonal chemistry, said use being based actually on the assumption that it cannot interfere with biochemical processes in ‘living systems’. Examples of prior art documents describing click-chemistry in protein/peptide synthesis include WO2012/36551; WO2011/161545; Davis et al. (Tetrahedron, 68, no. 4 (2011-11-28), p. 1029-1051); Haridas et al. (Tetrahedron, 67, no. 10 (2011-01-08), p. 1873-1884); Fekner et al. (Chembiochem, 12, no. 1 (2010-12-15), p. 21-33); Gasser et al. (Inorganic Chemistry, 48, no. 7, (2009-04-06), p. 3157-3166). Click-chemistry reagents disclosed in these prior art documents include Ub74-propargylamide, Ub75-propargylamide, Ub76-propargylamide, dipeptide alkyne-Leu-Leu-NH₂, pentapeptide alkyne-Leu-Leu-Phe-Leu-Val-N₃, Ac-Tyr-Gly-Gly-Phe-Leu-Prop (Ac-Enk-Prop) and Ac-Tyr-Gly-Pgl-Phe-Leu-NH₂ (Enk(Pgl)-NH₂) and alkyne truncated lysine dendrons, in particular (Boc protected) Lys-Lys(Lys)-Prop and (Boc protected) Lys-Lys(Lys)-Lys(Lys(Lys)-Lys)-Prop. None of the cited documents disclose or even suggest reactivity or activity of these click-chemistry reagents in biochemical process.

Contrary to the understanding that C-terminal alkynes as commonly used in click-chemistry reagents will not interfere with biochemical process in living systems, the present inventors have now found that even a non-strained terminal alkyne can react with reactive thiols, such as those found in active sites of cysteine proteases. For example, when positioned at the C-terminus of ubiquitin, a propargyl moiety can react with the active site of deubiquitinating enzymes (DUBs). The propargyl forms a covalent construct with the active site thiol. This reaction, which proceeds via a yet unknown mechanism, is very selective and the alkyne containing reagent is very reactive towards these proteases.

Ubiquitin-propargyl reacts with all classes of cysteine deubiquitinating enzymes, so-called DUBs, (USPs, UCHs, OTUs, Joseph-disease DUBs). Addition of propargyl to ubiquitin-like modifiers, such as SUMOs, FATT10, Nedd8, Urm1, Ufm1 etc., has been shown to result in similar reactivity towards the active site cysteine residue of the corresponding proteases. Small molecule truncations of the SUMO C-terminus covalently modify the active sites of SUMO specific proteases.

Without wishing to be bound by any theory, it is hypothesized that various strategies are feasible to develop cysteine protease capturing agents in accordance with this invention. Each strategy is based on the modification of a cleavage fragment of a cysteine protease substrate by introduction of a propargyl group, resulting in an agent that is still capable of being recognized by the corresponding protease, resulting in exposure of the active site cysteine side chain to the alkyne group of the propargyl moiety. Accordingly, substances are obtained capable of selectivity and irreversibly capturing a cysteine protease.

When a DUB cleaves an amide bond, it does so between a carboxy-terminal end of the ubiquitin and the lysine side chain of the other protein. As will be illustrated in the examples, the present inventors produced C-terminal modified ubiquitins that are still recognized by the DUB resulting in the formation of a covalent bond between the alkyne group of the propargyl moiety and the thiol group at the active site of the cysteine protease

Without wishing to be bound by any theory, it is also considered that, in the case of cysteine proteases cleaving amide bonds within the protein backbone, the introduction of a propargyl moiety at the C-terminal or N-terminal end of the respective fragments resulting from protease cleavage, will similarly result in substances capable of selectively capturing the corresponding protease.

The present inventors also have developed various propargyl-analogues, having various substitutions, such as alkyne reactivity tuning groups, which analogues can also suitably be used to modify protease substrate cleavage fragments, in accordance with this invention.

These and other aspects and embodiments of the invention will become apparent to those skilled in the art on the basis of the following detailed description and the experimental work described in the subsequent section.

### DETAILED DESCRIPTION OF THE INVENTION

Hence, in a first aspect, the present invention concerns cysteine protease capturing agents, especially in the form of peptides or peptide mimetics, comprising the N-terminal or C-terminal cleavage fragment of a cysteine protease substrate, characterized in that said fragment is modified by the addition of a propargyl moiety or analogue thereof capable of interacting with active site free thiol group of the cysteine protease.

A cysteine protease substrate may be a linear amino acid sequence or a non-linear protein conjugate comprising two (or more) linear amino acid sequences conjugated through an isopeptide bond, e.g. between a C-terminal carboxylic acid group and an epsilon amine of a lysine residue. As will be understood by those skilled in the art, a protein that is a substrate or target for a protease contains a specific sequence of amino acids that results in recognition and cleavage by the protease. Said sequence is referred to herein as ‘recognition sequence’ or ‘cleavage sequence’. A protease will cleave a specific amide bond within the substrate, which may be a linear amide bond or an isopeptide bond, resulting in two ‘fragments’. Within the context of this invention, this specific amide bond is referred to as the ‘cleavage site’ and the fragments resulting from cleavage by the protease are referred to as ‘cleavage fragments’.

The term ‘N-terminal cleavage fragment’ refers to the fragment containing the primary amine group that contributes to the amide that is cleaved by the protease in the corresponding substrate protein. This may also be referred to as the N→C cleavage fragment. In this designation the cleavage site is taken as the point of reference, meaning that the N-terminal site of the fragment contributes to the amide cleaved by the protease in the corresponding substrate protein. Similarly the term ‘C-terminal cleavage fragment’ refers to the fragment containing a terminal carboxylic acid group that contributes to the amide that is cleaved by the protease in the corresponding substrate protein. This may also be referred to as the C→N cleavage fragment. In this designation the cleavage site is taken as the point of reference, meaning that the c-terminal site of the fragment contributes to the amide cleaved by the protease in the corresponding substrate protein.

For ease of reference, the amino acid residues in the protease substrate and, hence, the corresponding fragments, are identified herein based on their position in the protein backbone relative to the cleavage site. In the context of the present invention the amino acid positions of the N-terminal fragment are designated 1, 2, 3, . . . , p, wherein 1 denotes the position adjacent to the cleavage site. The amino acids at these positions are designated a¹, a², a³, . . . , a^{p}, wherein a¹ is thus used to denote the amino acid containing the terminal amine group that contributed to the cleaved amide in the corresponding (natural) cysteine protease substrate. Similarly, the amino acid positions of the C-terminal fragment are designated −1, −2, −3, . . . , −p, wherein −1 denotes the position adjacent to the cleavage site and the amino acid residues are designated a⁻¹, a⁻², a⁻³, . . . , a^{-p}, wherein a⁻¹ denotes the amino acid residue containing the carboxylic acid group that contributes to the cleaved amide in the corresponding (natural) cysteine protease substrate.

In case the cysteine protease cleaves an isopeptide bond, typically between the C-terminal carboxyl group of one fragment and an amino acid side chain amine group of the other fragment, as is the case for e.g. deubiquitinating proteases, suitable capturing agents for the cysteine protease may be based on the isopeptide cleavage fragment, which is the fragment comprising the amino acid residue contributing to the isopeptide bond in the corresponding protease substrate. In such embodiments, the respective cleavage fragments will be referred to herein as the ‘C-terminal cleavage fragment’, following the definitions and designations described in the foregoing, and the ‘isopeptide cleavage fragment’.

Specific embodiments of the invention concern truncated versions of the cleavage fragments of the invention. It will be understood by those skilled in the art that, for maintaining the capability of the cleavage fragment to be recognized by the cysteine protease active site, truncations are limited to the terminal part of the fragments distant from the cleavage site of the corresponding protease substrate. The length of any truncation is not particularly limited provided that the remaining propargyl modified amino acid sequence is still capable of being recognized by the active site of the corresponding cysteine protease. In an embodiment of the invention, truncated versions of the cleavage fragments described herein are provided having a length of at least 2, at least 3, at least 4, at least 5, at least 6, at least 7, at least 9, at least 10, at least 15, at least 20 or at least 25 amino acid residues. In another preferred embodiment of the invention the fragment is the full length cleavage fragment of the corresponding protease substrate. In another embodiment, the fragment is a truncated version containing at least 50%, at least 60%, at least 70%, at least 80%, at least 85%, at least 90%, at least 92.5%, at least 95%, at least 97%, at least 98% or at least 99% of the amino acid sequence of said full length cleavage fragment.

As described above, the cleavage fragment as described herein before is modified by the by the introduction of a propargyl moiety or analogue thereof. Propargylamine as well as propargylic acid are commercially available. Propargylamine can conveniently be used to modify a terminal carboxylic acid group and propargylic acid can conveniently be used to modify a terminal amine group and/or an amine group in an amino acid side chain, using basic peptide synthesis chemistry. In particular, as will be illustrated in the examples, propargylamine can be attached chemically to the C-terminus of Ubiquitin lacking its C-terminal glycine residue, or a fragment thereof by either linear chemical synthesis followed by condensation of a ubiquitin derivative with propargylamine or by chemical ligation of propargylamine onto a ubiquitin75 thioester obtained by intein chemistry. Similarly, appropriate alkyne containing moieties, such as 4-butynoic acid or 4-butyn-1-amine can be attached to an N-terminal amine or an amino acid side chain amine by these same methods. Hence the skilled person is able to produce the capturing agents of this invention by e.g. by first obtaining a suitable peptide sequence, e.g. using conventional techniques such as solid phase peptide synthesis, and subsequently ligating the propargyl moiety as described here.

As indicated above, instead of the propargyl moiety, an analogue can be introduced. In accordance with the invention an analogue of propargyl is typically understood to encompass any variant of the basic propargyl moiety, wherein the reactivity of the alkyne group towards free thiol is retained or improved and wherein the alkyne group is not spatially hindered or constrained. In particularly preferred embodiments of the invention, certain functional groups can be introduced as substituents of the propargyl moiety, which increase the reactivity of the alkyne group towards free thiol. Suitable examples include halogen moieties, halogenated alkyl moieties, especially fluorine and/or fluorinated alkyl moieties.

The propargyl moiety or analogue thereof, typically, is introduced by substitution of the amino acid residue a¹ or a⁻¹. A structure is accordingly obtained having a terminal alkyne bond exactly at the position of the carbonyl double bond (at the carbon atom adjacent to the a-carbon atom) of amino acid a¹ or a⁻¹ of the corresponding ‘natural’ cleavage fragment, i.e. when the capturing agent and the ‘natural’ cleavage fragment are projected over one another. For instance, the experimental part below, describes ‘substitution’ of the C-terminal glycine residue of ubiquitin with a propargyl amine moiety.

Hence, in one embodiment, the invention concerns a cysteine protease capturing agent comprising the modified C-terminal portion of the C→N cleavage fragment of a cysteine protease substrate, wherein the cysteine protease capturing agent is represented by formula (I):

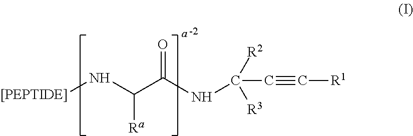

wherein:
R¹ represents hydrogen or a substituent selected from —F, —CF₃, —CHF₂, —CH₂F, —Cl, —CCl₃, —CHCl₂ and —CH₂Cl;
R^{a} represents an amino acid side chain identical to the amino acid side chain of the corresponding amino acid of the cysteine protease substrate;
R² and R³are independently selected from the group consisting of hydrogen, —F, —CF₃, —CHF₂, —CH₂F, —Cl, —CCl₃, —CHCl₂ and —CH₂Cl or one of R² and R³ represents a natural amino acid side chain, preferably the amino acid side chain of a⁻¹, while the other represents hydrogen; and
[PEPTIDE] represents a peptide chain comprising an amino acid sequence corresponding to a^{-p}-a⁻³; or an N-terminally truncated variant thereof having a length of at least 2 amino acid residues; or a homologue or conjugate thereof;
wherein a^{#} indicates the amino acid residue position in the corresponding intact cysteine protease substrate relative to the cleavage site thereof, a¹ and a⁻¹ being defined as the amino acid residues adjacent to the cleavage site; and wherein p represents an integer equal to the total number of amino acids of the C→N cleavage fragment of the cysteine protease substrate.

In another embodiment of the invention a cysteine protease capturing agent is provided comprising the modified N-terminal portion of the N→C fragment of the cysteine protease cleavage sequence of a cysteine protease substrate, wherein the cysteine protease capturing agent is represented by formula (II):

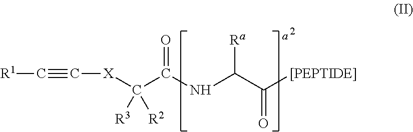

wherein:
R¹ represents hydrogen or a substituent selected from —F, —CF₃, —CHF₂, —CH₂F, —Cl, —CCl₃, —CHCl₂ and —CH₂Cl;
R^{a} represents an amino acid side chain identical to the amino acid side chain of the corresponding amino acid of the cysteine protease substrate;
R² and R³are independently selected from the group of hydrogen, —F, —CF₃, —CHF₂, —CH₂F, —Cl, —CCl₃, —CHCl₂ and —CH₂Cl or one of R² and R³ represents a natural amino acid side chain, preferably the amino acid side chain of a⁻¹, while the other represents hydrogen;
—X— represents a covalent bond or a moiety selected from —NH— and —CR⁴R⁵-, wherein R⁴ and R⁵ are independently selected from the group consisting of hydrogen, —F, —CF₃, —CHF₂, —CH₂F, —Cl, —CCl₃, —CHCl₂ and —CH₂Cl; and
[PEPTIDE] represents a peptide chain having an amino acid sequence corresponding to a³-a^{q}; or a C-terminally truncated variant thereof having a length of at least 2 amino acid residues; or a homologue or conjugate thereof;
wherein a^{#} indicates the amino acid residue position in the corresponding intact cysteine protease substrate relative to the cleavage site thereof, a¹ and a⁻¹ being defined as the amino acid residues adjacent to the cleavage site; and wherein q represents an integer equal to the total number of amino acids of the N→C cleavage fragment of the cysteine protease substrate.

In the above formulae (I) and (II), R¹referably represents hydrogen, —F or —CF₃, most preferably hydrogen.

In the above formulae (I) and (II), R² preferably represents hydrogen, —F or —CF₃, most preferably hydrogen.

In the above formulae (I) and (II), R³ preferably represents hydrogen, —F or —CF₃, most preferably hydrogen.

In one particularly preferred embodiment, one of -R² and -R³ represents an amino acid side chain, preferably the amino acid side chain of amino acid a⁻¹ (for formula (I)) or a¹ (for formula (II)) of the corresponding ‘natural’ protease substrate, while the other represents hydrogen.

In the above formula (II), R⁴ preferably represents hydrogen, —F or —CF₃, most preferably hydrogen.

In the above formula (II), R⁵ preferably represents hydrogen, —F or —CF₃, most preferably hydrogen.

In the above formula (II), X preferably represents —NH—.

In an embodiment of the invention at most one of R²-R⁵ in formulae (I) and/or (II) does not represent hydrogen.

In a preferred embodiment of the invention all of R²-R⁵ represent hydrogen.

In an embodiment all of R¹-R³ or all of R¹-R⁵ represent hydrogen.

As will be clear from the foregoing ‘R^{a}’ is used to refer to an amino acid side chain, typically an amino acid side chain of one of the naturally occurring amino acids, most preferably a side chain of an amino acid selected from the group consisting of Histidine; Alanine; Isoleucine; Arginine; Leucine; Asparagine; Lysine; Aspartic acid; Methionine; Cysteine; Phenylalanine; Glutamic acid; Threonine; Glutamine; Tryptophan; Glycine; Valine; Proline; Selenocysteine; Serine; and Tyrosine. As will be clear from the definition and explanations in the foregoing, R^{a} typically corresponds to the side chain of the amino acid residue at the respective position in the corresponding (natural) cysteine protease substrate, as indicated by a#.

As will be clear from the explanation and definitions above [PEPTIDE], in formulae (I) and (II), typically represents an amino acid sequence, identical to the corresponding portion of the naturally occurring cysteine protease substrate. In this context ‘corresponding portion’ means the amino acid sequence found in the naturally occurring cysteine protease substrate at the same position relative to the cleavage site. For example, if the cysteine protease is a deubiquitinating enzyme, a^{-p}-a⁻¹ in formula (I) represent the entire naturally occurring ubiquitin sequence and [PEPTIDE] in formula (I) thus typically defines said entire ubiquitin sequence minus the two C-terminal amino acids. Truncated versions of these amino acid sequences, homologues of these amino acid sequences and/or conjugates comprising these amino acid sequences are also encompassed by the meaning of [PEPTIDE], provided that the resulting agent is still capable of being recognized by and interacting with the active site of the cysteine protease.

Hence, in an embodiment [PEPTIDE] represents truncated versions of the corresponding portions of the ‘wild-type’ cysteine protease substrate, with the proviso that the resulting agent is still capable of being recognized by and interacting with the active site of the cysteine protease. Preferably, in the above formulae [PEPTIDE] represents an amino acid sequence having a length of at least 1, at least 2, at least 3, at least 4, at least 5, at least 6, at least 7, at least 9, at least 10, at least 15, at least 20 or at least 25 amino acid residues.

In an embodiment [PEPTIDE] represents homologues of the corresponding portions of the ‘wild-type’ cysteine protease substrate, with the proviso that the resulting agent is still capable of being recognized by and interacting with the active site of the cysteine protease. The term ‘homologue’ is used herein in its common meaning, as referring to polypeptides which differ from the reference polypeptide, by minor modifications, but which maintain the basic polypeptide and side chain structure of the reference peptide. Such changes include, but are not limited to: changes in one or a few amino acid side chains; changes in one or a few amino acids, including deletions, insertions and/or substitutions; changes in stereochemistry of one or a few atoms; additional N- or C-terminal amino acids; and/or minor derivatizations, including but not limited to: methylation, glycosylation, phosphorylation, acetylation, myristoylation, prenylation, palmitation, amidation and/or addition of glycosylphosphatidyl inositol. Non-naturally occurring mutants of particular interest furthermore include mutants comprising certain insertions and/or substitutions that create ligation handles, especially the substitution of lysine with d-thiolysine, δ-selenolysine, γ-thiolysine, γ-selenolysine (all as described in co-pending patent application no. PCT/NL2010/050277) or δ-azido ornithine or the substitution of leucine with photoleucine. As used herein, a homologue or analogue has either enhanced or substantially similar functionality as the naturally occurring polypeptide. A homologue herein is understood to comprise a polypeptide having at least 70%, preferably at least 80%, more preferably at least 90%, still more preferably at least 95%, still more preferably at least 98% and most preferably at least 99% amino acid sequence identity with the reference polypeptide, when optimally aligned, such as by the programs GAP or BESTFIT using default parameters, and is still capable of eliciting at least the immune response obtainable thereby. Generally, the GAP default parameters are used, with a gap creation penalty=8 and gap extension penalty=2. For proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752, USA. Alternatively percent similarity or identity may be determined by searching against databases such as FASTA, BLAST, etc. In an embodiment [PEPTIDE] represents conjugates of the corresponding portions of the ‘wild-type’ cysteine protease substrate with another peptide or protein, which may be conjugated in a linear or non-linear fashion, with the proviso that the capability of the resulting agent to be recognized by and interacting with the active site of the cysteine protease is retained. Such conjugates may be used to introduce or affect chemical or biological functionality, e.g. cell permeability enhancement, proteasome targeting, introduction of sites for directed chemical modifications (introduction of a so-called ‘ligation handle’), affinity tagging, etc. Preferred examples include addition of cell penetration enhancing peptide sequences such as (D-Arg)8, Tat and penetratin; addition of affinity tag peptide sequences, such as HA and His6; addition of a proteasome targeting handle such as L4; and substitution of N- or C-terminal residues.

Cysteine proteases, in accordance with this invention, are proteases having a catalytic mechanism involving nucleophilic cystein thiol in the enzyme's active cite. Suitable examples of cysteine proteases in accordance with this invention typically include cathepsin B; cathepsin C; cathepsin F; cathepsin H; cathepsin K; cathepsin L; cathepsin L2; cathepsin O; cathepsin S; cathepsin W; cathepsin Z; cathepsin J; cathepsin M; cathepsin Q; cathepsin Q2; cathepsin Q2-like; cathepsin R; cathepsin-1; cathepsin-2; cathepsin-3; cathepsin-6; cathepsin-7-like; tubulointerstitial nephritis antigen; TINAG related protein; testin; testin-2; testin-3; bleomycin hydrolase; calpain 1; calpain 2; calpain 3; calpain 5; calpain 6; calpain 7; calpain 7-like; calpain 8; calpain 9; calpain 10; calpain 11; calpain 12; calpain 13; calpain 14; calpain 15/Solh protein; ubiquitin C-terminal hydrolase 1; ubiquitin C-terminal hydrolase 3; ubiquitin C-term. hydrolase BAP1; ubiquitin C-terminal hydrolase 5; ubiquitin C-terminal hydrolase 4; legumain; hGPI8; caspase-1; caspase-2; caspase-3; caspase-4/11; caspase-5; caspase-6; caspase-7; caspase-8; caspase-9; caspase-10; caspase-12; caspase-14; paracaspase; homologue ICEY; casper/FLIP; caspase-14-like; pyroglutamyl-peptidase I; pyroglutamyl-peptidase II; USP1; USP2; USP3; USP4; USP5; USP6; USP7; USP8; USP9X; USP9Y; USP10; USP11; USP12; USP13; USP14; USP15; USP16; USP17; USP17-like; USP18; USP19; USP20; USP21; USP22; USP24; USP25; USP26; USP27; USP28; USP29; USP30; USP31; NY-REN-60; VDU1; USP34; USP35; USP36; USP37; HP43.8KD; SAD1; USP40; USP41; USP42; USP43; USP44; USP45; USP46; USP47; USP48; USP49; USP50; USP51; USP52; USP53; USP54; DUB-1; DUB-2; DUB2a; DUB2a-like; DUB2a-like2; DUB6; BAP1; UCHL1; UCHL3; UCHL5; gamma-glutamyl hydrolase; Gln-PRPP amidotransferase; Gln-fructose-6-P transamidase 1; Gln-fructose-6-P transamidase 2; Gln-fructose-6-P transamidase 3; sonic hedgehog protein; indian hedgehog protein; desert hedgehog protein; sentrin/SUMO protease 1; sentrin/SUMO protease 2; sentrin/SUMO protease 3; sentrin/SUMO protease 5; sentrin/SUMO protease 5-like 1; sentrin/SUMO protease 6; sentrin/SUMO protease 7; sentrin/SUMO protease 8; sentrin/SUMO protease 9; sentrin/SUMO protease 11; sentrin/SUMO protease 12; sentrin/SUMO protease 13; sentrin/SUMO protease 14; sentrin/SUMO protease 15; sentrin/SUMO protease 16; sentrin/SUMO protease 17; sentrin/SUMO protease 18; sentrin/SUMO protease 19; separase;autophagin-1; autophagin-2; autophagin-3; autophagin-4; DJ-1; cezanne/OTU domain containing 7B; cezanne-2; A20, TNFa-induced protein 3; TRAF-binding protein domain; VCP(p97)/p47-interacting protein; Hin-1/OTU domain containing 4; asparagine-linked glycosylation 13 homolog; OTU domain containing-3; OTU domain containing-1; OTU domain containing-6A; OTUD2/YOD1; OTU domain containing 6B; CGI-77b; otubain-1; otubain-1 like; otubain-2; cylindromatosis protein; secernin-1; secernin-2; secernin-3; Ufm-1 specific protease 1; Ufm-1 specific protease 2; nasal embryonic LHRH factor; epithelial cell transforming sequence 2 oncogene-like; OTU domain containing-5; ataxin-3; ataxin-3 like; josephin-1; josephin-2; acid ceramidase; HetF-like; zinc finger CCCH-type containing 12A; zinc finger CCCH-type containing 12B; zinc finger CCCH-type containing 12C; zinc finger CCCH-type containing 12D; NYN domain and retroviral integrase containing; KHNYN KH and NYN domain containing; NEDD4 binding protein 1.

In accordance with the present invention the cysteine protease is preferably selected from the group of deubiquitinating proteases, cathepsins, calpains, caspases and SUMO proteases, preferably from the group of deubiquitinating proteases, SUMO protease, caspases and cathepsins, more preferably from the group of deubiquitinating proteases and SUMO proteases, and most preferably from the group of deubiquitinating proteases.

Accordingly, as will be understood by those skilled in the art, the cysteine protease substrate preferably is a protein targeted by these respective groups of cysteine proteases. In a preferred embodiment of the invention, a cysteine protease capturing agent represented by formula (I) is thus provided wherein a^{-p}-a⁻¹ represents ubiquitin (SEQ ID NO. 1). In another embodiment, a cysteine protease capturing agent represented by formula (I) is provided wherein a⁻¹-a^{-p} represents a non-natural ubiquitin variant selected from UbM1C (SEQ ID no. 2); HA-Ub (SEQ ID no. 3); His6-Ub (SEQ ID no. 4); (D-Arg)8-Ub (SEQ ID no. 5);; penetratin-Ub (SEQ ID no. 6); Tat-Ub (SEQ ID no. 7); UbM1(OrnN₂) (SEQ ID no. 8); UbK6(OrnN₂) (SEQ ID no. 9); UbK11(OrnN₂) (SEQ ID no. 10); UbK27(OrnN₂) (SEQ ID no. 11); UbK29(OrnN₂) (SEQ ID no. 12); UbK33(OrnN₂) (SEQ ID no. 13); UbK48(OrnN₂) (SEQ ID no. 14); UbK63(OrnN₂) (SEQ ID no. 15);, UbK6(δ-thioK) (SEQ ID no. 16), UbK11(δ-thioK) (SEQ ID no. 17); UbK27(δ-thioK) (SEQ ID no. 18); UbK29(δ-thioK) (SEQ ID no. 19); UbK33(δ-thioK) (SEQ ID no. 20); UbK48(δ-thioK) (SEQ ID no. 21); and UbK63(δ-thioK) (SEQ ID no. 22), UbK48(y-thioK) (SEQ ID no. 23), UbL43photoLeu (SEQ ID no. 24), UbL71photoLeu (SEQ ID no. 25) and UbL73photoLeu (SEQ ID no. 26), all as defined in table 1 below.

In accordance with another embodiment of the invention, a cysteine protease capturing agent represented by formula (I) is provided wherein a^{-p}-a⁻¹ represents a ubiquitin-like modifier, such as SUMO 1 (SEQ ID no. 27); SUMO 2 (SEQ ID no. 28); SUMO 3 (SEQ ID no. 29); SUMO 4 (SEQ ID no. 30); Nedd8 (SEQ ID no 31); FATT10 (SEQ ID no 32); ISG15 (SEQ ID no. 33); Urm1 (SEQ ID no. 34); or Ufm1 (SEQ ID no. 35).

In an embodiment of the invention a^{-p}-a⁻¹ does not represent ubiquitin, enkephalin or a lysine dendron.

In addition to variations in the amino acid sequence, the here described invention also entails cysteine protease capturing agents comprising a derivative of the modified above defined modified cleavage fragments, typically comprising a ligand coupled to an amino acid side chain thereof and/or the N-terminus and/or the C-terminus thereof. As used herein the terms ‘derivative’ thus refer to products comprising a modified cleavage fragment as defined herein before, further comprising one or more ligands derivatized to the C-terminal carboxyl group, the N-terminal amine group and/or an amino acid side chain. Such ligands may, in principle, be of any nature, including peptides or proteins, lipids, carbohydrates, polymers and organic or inorganic agents. The introduction of the ligand typically introduces or affects a particular biological or chemical function. Particularly interesting examples include the introduction of detectable labels and tags, introduction of electrophilic traps, introduction of chemical ligation moieties, etc. Hence, in a preferred embodiment, a method as defined herein before is provided, wherein said derivative comprises a ligand selected from the group of fluorophores, affinity labels, biophysical labels, chelating agents, complexing agents and epitope tags, such as fluorescein (formula (E)), TAMRA (formula (F)) or DOTA (formula (G). Those skilled in the art will be familiar with these types of ligands and their introduction at a desired site can be accomplished using reagents and conditions that are generally known.

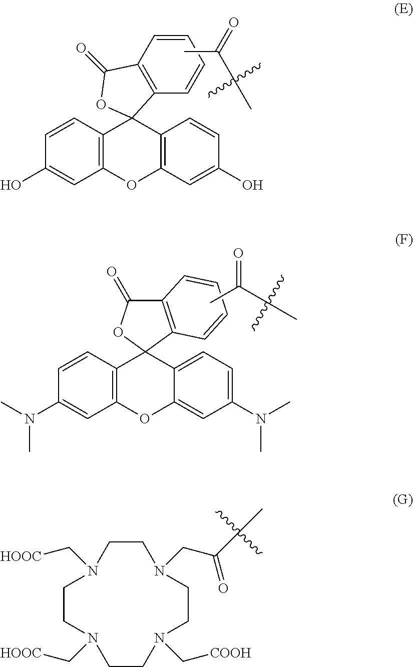

The present invention also entails cysteine protease capturing agents in the form of peptide mimetics comprising a spatial arrangement of (re)active chemical moieties and/or functional groups that resembles the three-dimensional arrangement of active and/or functional groups of any one of the peptide cysteine protease capturing agents defined herein before, wherein the peptide mimetic comprises the propargyl or modified propargyl moiety of any one of said peptide cysteine protease capturing agents and wherein said peptide mimetic is capable of being recognized by and interacting with the active site of the cysteine protease.

A peptide mimetic (peptidomimetic) is a molecule that mimics the biological activity of a peptide, yet is no longer peptidic in chemical nature. By strict definition, a peptidomimetic is a molecule that no longer contains any peptide bonds, i.e., amide bonds between amino acids; however, in the context of the present invention, the term peptide mimetic and also the term peptidomimetic are intended to include molecules that are no longer completely peptidic in nature, such as pseudo-peptides, semi-peptides and peptoids. Whether completely or partially nonpeptide, peptidomimetics according to the present invention provide a spatial arrangement of (re)active chemical moieties and/or functional groups that closely resembles the three-dimensional arrangement of active and/or functional groups in the peptide on which the peptidomimetic is based. The techniques of developing peptidomimetics are conventional. Thus, non-peptide bonds that allow the peptidomimetic to adopt a similar structure to the original peptide can replace peptide bonds. Replacing chemical groups of the amino acids with other chemical groups of similar structure can also be used to develop peptidomimetics. Conventional approaches allow for the development of peptidomimetics in accordance with this invention.

In an embodiment of the invention, the cysteine protease capturing agent is not Ub74-propargylamide (wherein ‘Ub74’ refers to an amino acid chain comprising amino acids 1-74 of the natural Ub sequence), Ub75-propargylamide, Ub76-propargylamide, alkyne-Leu-Leu-NH₂ (1), alkyne-Leu-Leu-Phe-Leu-Val-N₃ (2), Ac-Tyr-Gly-Gly-Phe-Leu-Prop (wherein Prop means propargylamine) (3), Ac-Tyr-Gly-Pgl-Phe-Leu-NH₂ (wherein Pgl means propargylglycine) (4), Boc-protected or unprotected Lys-Lys(Lys)-Prop (5) or Boc protected or unprotected Lys-Lys(Lys)-Lys(Lys(Lys)-Lys)-Prop (6), as depicted below.

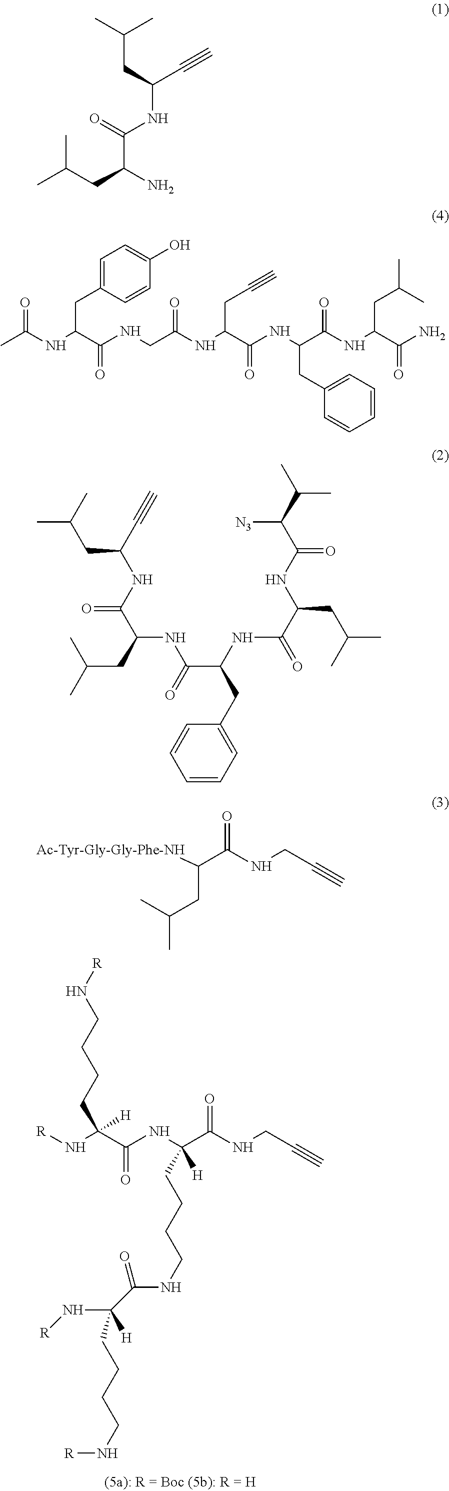

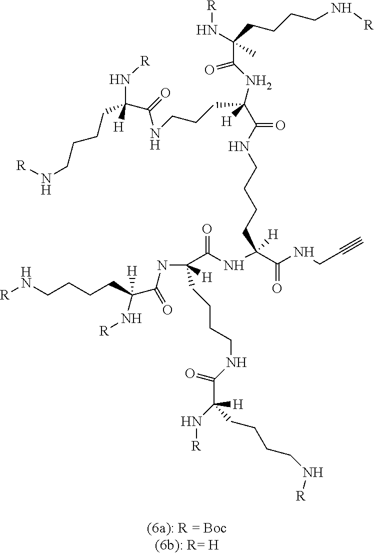

Another aspect of the present invention concerns a method of producing a cysteine protease capturing agent comprising the steps of: i) identifying a substrate for the cysteine protease; ii) obtaining a cleavage fragment resulting from cleavage of the naturally occurring substrate by the cysteine protease; and iii) modifying the cleavage fragment by introduction of a propargyl moiety capable of interacting with the thiol side chain of the cysteine residue present in the active site of the cysteine protease. As will be understood by those skilled in the art, the substrate for the cysteine protease, typically will be a/the natural substrate for the cysteine protease. Furthermore, as will be clear from the foregoing, the method may comprise additional steps of modifying the cleavage fragment, e.g. by truncations, derivatizations, conjugations, amino acid deletions, insertions or substitutions, etc., with the proviso that the capability of the resulting agent to be recognized by and interacting with the active site of the cysteine protease is retained by said modification.

As will be understood, particularly preferred features described here above in relation to the capturing agents, are of equal interest to the method of producing cysteine protease capturing agents.

Another aspect of the invention concerns cysteine protease capturing agents obtainable by the afore-defined method.

Another aspect of the present invention concerns the use of the cysteine protease capturing agents as defined in any of the foregoing as a medicament, a diagnostic agent and/or as biochemistry research tool.

As will be understood by one skilled in the art the substances of the present invention can be used to capture their corresponding cysteine protease, e.g. from a highly complex biological matrix, which can be of particular use in both diagnostics and fundamental research.

Hence, the invention, in one aspect, also provides a method of capturing a cysteine protease from a biological sample, said method comprising the steps of: a) providing said sample comprising a cysteine proteases; b) combining the sample with a corresponding cysteine protease capturing agent of this invention, wherein said cysteine protease capturing agent is conjugated to a chelating agent, a complexing agent, an epitope tag or a solid phase, which allows for or results in immobilization of the cysteine protease capturing agent; c) subjecting the sample to conditions that allow for selective binding of the cysteine protease to the cysteine protease capturing agent; d) separating the sample from the immobilized cysteine protease capturing agent. Immobilization of cysteine protease capturing agents, which take the form of (conjugated/derivatized) peptides or peptide mimetics, can be achieved using various techniques familiar to those skilled in the art. Depending on the choice of immobilization technique the above-described method may comprise the additional step of combining the sample comprising the cysteine protease capturing agent with a solid phase capable of immobilizing the cysteine protease capturing agent, prior to any one of steps a), b), c) or d). If the immobilization step is done after step b), as will be understood, a technique is to be selected involving selective trapping under condition which do not affect other components of the biological sample. Hence, it will be appreciated that a preferred embodiment of the method comprises immobilization of the cystein protease capturing agent prior to step b).

As will be understood by those skilled in the art, immobilization of the cysteine protease capturing agent can be accomplished in various ways. In one embodiment of the invention, the cysteine protease capturing agent is immobilized using CNBr-activated sepharose.

In an embodiment of the invention, the above method involves the use of a cysteine protease capturing agent that is conjugated/derivatized with a detection label as defined herein above, wherein the method comprises one or more additional steps of quantifying the binding of cysteine protease to the cysteine protease capturing agent.

As explained herein before the present cysteine protease capturing agents bind their corresponding cysteine protease in a selective and highly irreversible manner, allowing for stringent washing conditions, which makes the present method highly effective.

The above method may be used in research concerning any biological process involving the action of a cysteine protease and/or in diagnosing any condition or disease involving the action of a cysteine protease.

Since the present cysteine protease capturing agents are capable of selective and highly irreversible binding of their corresponding cysteine protease, it is also envisaged that the cysteine protease capturing agents have utility as (competitive) protease inhibitors or antagonistic agents in various therapeutic methods. Typically such therapeutic methods are aimed at the treatment or prevention of a condition or disease, involving the action of a cysteine protease.

Conditions or diseases involving the action of cysteine proteases may include auto immune diseases, cancer (metastatic and non-metastatic), infections and lysosomal storage diseases.

The invention, in further aspects, provides the use of a cysteine protease capturing agent as defined in the foregoing as an inhibitor or antagonist of a corresponding cysteine protease; a method of inhibiting cysteine protease activity by exposing the cysteine protease to a corresponding capturing agent as defined herein before; and the cysteine protease capturing agent for use in any such method.

Thus, the invention has been described by reference to certain embodiments discussed above. It will be recognized that these embodiments are susceptible to various modifications and alternative forms well known to those of skill in the art.

Many modifications in addition to those described above may be made to the structures and techniques described herein without departing from the spirit and scope of the invention. Accordingly, although specific embodiments have been described, these are examples only and are not limiting upon the scope of the invention.

Furthermore, for a proper understanding of this document and in its claims, it is to be understood that the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

All patent and literature references cited in the present specification are hereby incorporated by reference in their entirety.

The following examples are offered for illustrative purposes only, and are not intended to limit the scope of the present invention in any way.

### Example 1—Reactivity of Alkynes with Active-Site Cysteines—Novel Active-Site Directed Probes to Study Deubiquitination

Bioorthogonal reactions, such as “click reactions” have proven powerful tools to study protein function. However, the bioorthogonality of various click chemistries is not complete.

Here it is demonstrated that inert bioorthogonal non-strained alkynes can react with nucleophilic thiol residues, such as those found in active sites of cysteine proteases.

### Materials and Methods

### General:

General reagents were obtained from Sigma Aldrich, Fluka and Acros and used as received. Peptide synthesis reagents were purchased from Novabiochem. LC-MS measurements were performed on a system equipped with a Waters 2795 Separation Module (Alliance HT), Waters 2996 Photodiode Array Detector (190-750 nm), Phenomenex Kinetex C18 (2.1×50, 2.6 μm) and LCT™ Orthogonal Acceleration Time of Flight Mass Spectrometer. Samples were run using 2 mobile phases: A=1% CH₃CN, 0.1% formic acid in water and B=1% water and 0.1% formic acid in CH₃CN. Data processing was performed using Waters MassLynx Mass Spectrometry Software 4.1 (deconvolution with Maxent I function).

### Production of Recombinant UCH-L3:

Recombinant UCH-L3 was expressed and purified as described previously (C. N. Larsen, J. S. Price, K. D. Wilkinson, Biochemistry 35, 6735 (1996)). Analysis by LC-MS showed a single form of the purified protein.

### Synthesis of Ubiquitin₇₅-Propargylamide:

Ubiquitin 1-75 was synthesised using the method described in F. El Oualid et al., Angewandte Chemie International Edition 49, 10149 (2010).

Briefly, Ubiquitin-75 was synthesized on TentaGel® R TRT-Gly Fmoc obtained from Rapp Polymere GmbH (RA 1213) and its identity confirmed by treating a small amount of resin with TFA cleavage cocktail (92,5% TFA, 5% H₂O, 2,5% Triisopropylsilane), followed by LC-MS analysis. The N-terminal Fmoc-group was removed by treatment with 20% piperidine in N-Methyl-2-pyrrolidone (NMP) (3×10 minutes incubation). After treatment the resin was washed 3 times with NMP followed by 3 washes with Methylene Chloride (DCM) to remove traces of NMP.

The resin was then incubated for 30 minutes with 2 bed volumes of hexafluoroisopropanol/dichloromethane mixture (2:8) to afford a DCM soluble protected ubiquitin polypeptide. After drying the solid it was taken up in 5 ml DCM, to which was added 65 mg PyBOP (125 μmole, 5 eq), 17.4 μL triethylamine (125 μmole, 5 eq) and propargylamine (250 μmole, 10 eq). The reaction mixture was stirred at room temperature overnight before concentrating it in vacuo. Residual propargylamine was removed by co-evaporation with DCM and toluene and the resulting off-white solid was dried overnight in high vacuum. This is the point where an N-terminal modification can be introduced.

The propargylated ubiquitin was then deprotected using a mixture of trifluoroacetic acid, water and triisopropylsilane (95:3:2), and precipitated in cold diethyl ether/pentane (3:1). The precipitated crude product was collected by centrifugation (1000 g, 5 minutes) and washed 3× with cold diethyl ether.

### Purification of Ubiquitin₇₅-Propargylamide (Ub₇₅-Prg)

Ubiquitin₇₅-propargylamide was purified by cation exchange chromatography followed by preparative reverse phase HPLC as described previously in F. El Oualid et al., Angewandte Chemie International Edition 49, 10149 (2010).

TAMRA Labeled Ubiquitin with C-Terminal Propargyl Amine (TMR-Ub-prg)

C-terminal propargylamine was first introduced according to the procedure described above. Before the final drying step and deprotection as described above, the protected version of Ubiquitin₇₅-propargylamide was dissolved in DCM and extracted 4× with equal volume of 1 M KHSO₄ to remove traces of propargylamine. After drying the DCM layer over MgSO₄, solvent was removed in vacuo.

Tetramethylrhodamine (TAMRA, 56 mg, 125 μmole, 5 eq) was preactivated in anhydrous DMF by the addition of PyBOP (65 mg, 125 μmole, 5 eq) and triethylamine (17.5 μL, 125 μmole, 5eq). The reaction was incubated for 5 minutes prior to the addition of protected Ub-propargyl to the reaction mixture (25 μmole). After reacting overnight the solvent was removed under reduced pressure and the resulting deep purple solid was deprotected using a mixture of trifluoroacetic acid, water and triisopropylsilane (95:3:2), and precipitated in cold diethyl ether/pentane mixture (3/1). The precipitated crude product was collected by centrifugation (1000 g, 5 minutes) and washed 3× with cold diethyl ether prior to lyophilisation and subsequent purification as described above

### Fluorescence Polarization Assay of UCH-L3 Inhibition in Presence of Ubiquitin₇₅-Propargylamide.

The fluorescence polarization assay was performed as described previously in P. P. Geurink, F. El Oualid, A. Jonker, D. S. Hameed, H. Ovaa, Chembiochem 13, 293 (2012).

Reaction between Ubiquitin₇₅-Propargylamide and DUBs

General procedure: DUB was dissolved in PBS with 5 mM dithiothreitol to a concentration of 1 mg/mL. Ubiquitin₇₅-propargylamide was dissolved in DMSO to a concentration of 10 mg/ml. 1.2 molar equivalents of Ubiquitin₇₅-propargylamide were added from this stock solution to the DUB-solution and the reaction was allowed to proceed at 37 ° C. under gentle agitation for 30 minutes after which it was analysed by LC-MS and/or SDS-PAGE.

### Purification of the Complex and Analytical Data

After successful modification of the DUB the solution was buffer exchanged using sephadex G25 resin (PD-10, GE Healthcare) to Buffer A (50 mM Tris, pH 7.5, 5% Glycerol). The sample was applied on 6ml Resource Q cartridge using AKTA Purifier system. The complex was eluted using a shallow gradient running from 20-30% Buffer B (50 mM, pH 7.5; 5% Glycerol; 500 mM NaCl). Resulting fractions were analysed on SDS-PAGE and pooled according to purity.

Acid Cleavage of the Complex between Ubiquitin₇₅-Propargylamide and the DUBs

To a solution of Ubiquitin₇₅-propargylamide-UCH-L3 complex in PBS (3 mg/mL; 10 μL) was added a 3-fold concentrated solution of the tested acid. The reactions were incubated overnight and analyzed by SDS-PAGE chromatography and/or mass spectrometry.

### LC-MS Analysis of Thioradical-Mediated Cleavage Reactions.

UCH-L3 (8 mg/mL, 10 μL) and Ubiquitin₇₅-propargylamide (10 mg/mL in DMSO, 10 μL), were dissolved in PBS (79 μL) and DTT was added (1 M, 1 μL). The reaction was incubated for 30 minutes at 37° C. and was buffer exchanged into water by PD-10 column (GE Healthcare) and lyophilized overnight. The resulting white powder was dissolved in PBS (100 μL) and to 90 μL of this solution 10 μL of ethanethiol was added (143 μmole) and VA-044 to a final concentration of 10 mM. The reaction was shaken at 37° C. for three hours prior to LC-MS analysis.

### Labeling of Overexpressed DUBs in Cell Lysates

For overexpression of GFP-tagged DUBs in cells, wild type USP14, Otub1, Otub2, Otud1 and POH1 were subcloned from pDEST-cDNA constructs (Addgene) into the eGFP-C1 vector system (Clontech) at XhoI/EcoRI restriction sites. Mutagenesis of catalytic Cysteines was performed by standard protocols (Stratagene) using the following primers (sites of mutagenesis underlined): USP14-C114S forward-CTT GGT AAC ACT TCT TAC ATG AAT GCC, reverse-GGC ATT CAT GTA AGA AGT GTT ACC AAG; Otub1-C9/S forward-CCT GAC GGC AAC TCT TTC TAT CGG GC, reverse-GC CCG ATA GAA AGA GTT GCC GTC AGG; Otub2-C51S forward-GG GAT GGG AAC TCC TTC TAC AGG GCC, reverse-GGC CCT GTA GAA GGA GTT CCC ATC CC. DNA delivery into MelJuSo cells was performed in 60 mm tissue culture plates using Lipofectamine2000 (Invitrogen) according to manufacturer's instructions. 24 h following transfection, cells were harvested by scraping in 0.25 ml HR lysis buffer, and reactions were incubated for 30 min with agitation at 37° C. in the absence or presence of the probe (4 μg/reaction). Reactions were stopped by the addition sample loading buffer supplemented with (β-mercaptoethanol, followed by boiling for 10 min. Samples were resolved on 4-12% MOPS NU-PAGE Gels (Invitrogen) and probe reactivity was assessed by TAMRA fluorescence scanning as described above. Gels were then transferred onto Nitrocellulose membranes and immunoblotted using anti-GFP serum produced in rabbit and mouse anti-β-actin (Sigma-Aldrich). Immunoblots were visualized using a Licor Odyssey. The following fluorescent secondary antibodies purchased from LICOR were used: anti-mouse-680, anti-rabbit-800.

### Immobilization of Ubiquitin₇₅-Propargylamide on CNBr-Resin

Ub-Prg (10 mg, 1.2 μmole) or Ubiquitin were immobilized on 500 mg of cyanogen bromide (CNBr)-activated Sepharose 4b resin according to the manufacturer's protocol (GE Healthcare). Spectrophotometric analysis before and after coupling of the ubiquitin derivatives, indicated the coupling of 10 mg of either protein per gram of resin.

Pulldown of UCH-L3 from a Mixture of UCH-L3 and BSA

UCH-L3 and bovine serum albumin were dissolved in PBS to 4 mg/mL for either protein. 50 μL of the mixture was diluted with the indicated amounts of the above resin for 2 hours at 37° C. As a control Sepharose 4B modified with Ub-76 was included. After incubation the flowthroughs were analysed by SDS-PAGE (left figure).

### Washing and Retrieval of Immobilized UCH-L3

UCH-L3 and BSA were mixed as above and 100 μL of the above mixture was incubated with 50 mg of the Ub-prg-sepharose at 37° C. for 2 hours. The resin was then washed with 5% SDS in PBS (3×500 μL) and water (3×500 μL).

After washing the resin was incubated with 10% trifluoroacetic acid (TFA) in water for 3 hours. The TFA and water were removed under reduced pressure followed by lyophilization overnight. The resulting powder was resuspended in Nu-PAGE LDS sample buffer (4x concentrate, 100 μL) and boiled for 20 minutes. The insoluble resin-fraction was removed by centrifugation at 14,000 g prior to analysis by SDS-PAGE (right gel below).

Pulldown of DUBs from Cell Lysates

50 mg of the above resin was incubated with EL-4 lysate (1 mL, 3 mg/mL soluble protein in PBS+10 mM DTT +Roche protease inhibitor tablet) at 37° C. for 3 hours. After elution of the flowthrough, the resin was washed with PBS +1% Triton X-100 (3×10 mL), 2% CHAPS, 8 M Urea in PBS (6×10 mL) and water (6×10 mL).

Proteins were eluted by incubation of the resin with 10% trifluoroacetic acid (TFA) in water for 3 hours. The slurry of resin in TFA was dried under reduced pressure and lyophilized overnight. The resulting powder was resuspended in NuPAGE LDS-sample buffer (100 μL, Life Technologies) and incubated at 99° C. for 30 minutes. The insoluble fraction was removed by centrifugation (14,000 g, 10 minutes) and the supernatant was analyzed by SDS-PAGE.

### Analysis of Cleavage Conditions for Radical Pull Down Experiment

To a solution of Ub-prg-UCH-L3 complex in PBS (0.5 mg/mL; 80 μL) various amounts of thiol were added to the final concentrations indicated. Radical initiator VA-044 was added at the concentrations indicated and the reaction was shaken at 37° C. for 3 hours after which the reactions were analysed by non-reducing SDS-PAGE.

### Results

It is shown that Ub-Prg forms a bond with UCH-L3 that is stable to both denaturing LC-MS conditions (FIG. 1B) as well as denaturing SDS-PAGE (FIG. 1C). Pre-treatment of UCH-L3 with a general alkylating agent, iodoacetamide, prior to addition of Ub-Prg , abolished all reactivity with UCH-L3. The addition of a 1000-fold excess of propargylamine or free cysteine did not affect the outcome of the reaction (FIG. S1). The hypothesis of a non-radical mechanism was strengthened by complex formation in the presence of excess radical scavenger. To exclude the possibility that a contaminant in the preparation of Ub-Prg was active a titration of Ub-Prg against UCH-L3 was performed and it was found that the stoichiometry of the reaction was 1:1. These data indicated that a covalent linkage between the active site cysteine residue of UCH-L3 and Ub-Prg had formed; despite the fact that the terminal alkyne moiety to date was considered inert. The reaction proved selective for terminal alkynes, as allylamine, but-3-enylamine or propylamine modified ubiquitin derivatives proved resistant towards DUB-mediated modification.

The assumed reaction mechanism involves direct attack of the active site thiol on the quaternary carbon of the alkyne moiety to result in a thiovinylether (FIG. 2C). In this reaction scenario the quaternary alkyne moiety aligns with the Glycine76 caboxylate, the usual site for Ub deconjugation. The geometries of DUB active sites bound to Ub-aldehyde or Ub probes as seen in several crystal structures also support this hypothesis. This reaction would result in the formation of a single quaternary vinyl thioether, which would correspond to the mass of Ub-Prg plus the mass of the DUB.

Analysis of the complex formed by LC-MS (FIG. 1B) indeed confirmed this. Vinyl thioethers are also known to be acid labile, and incubation of the complex between Ub-Prg and the DUB UCH-L3 with various acids resulted in the cleavage of the complex. The observed mass for Ub-Prg after cleavage with dilute trifluoroacetic acid is 8565 Da, which corresponds to the mass of a hydrolyzed thioether. In addition, mutation of the active site cysteine to a serine residue abolished binding of the probe (FIG. 2B).

These data combined support the conclusion that a quaternary vinyl thioether reaction product is formed through condensation of active site cysteine nucleophiles and the triple bond in probe Ub-Prg.

DUB probe Ub-Prg was found to react in vitro with all cysteine protease DUBs tested, including OTU-domain containing DUBs that frequently prove inert towards modification with various DUB probes. Ubiquitin propargylamide proved unreactive towards other cysteine proteases tested including papain, the archetypal cysteine protease, and SENP-1, a hydrolase specific for the ubiquitin-like protein SUMO, while no reactivity towards the ubiquitin ligase E1 was observed (FIG. 2A).

After determining the scope of the reactivity of Ub-Prg, an N-terminally tetramethylrhodamine (TMR) labeled analogue of Ub-Prg (TMR-Ub-Prg, FIG. 1) was synthesized to analyze if the probe could be used to label DUBs in cell lysates. Labeling of active DUBs in lysates at endogenous levels with this fluorescent probe also led to clear labeling results allowing its use in activity profiling experiments (FIG. 2B). To further strengthen the notion that this probe is DUB-selective HeLa cells were transfected with a series of GFP-DUB fusions (FIG. 2B) and the capacity of TMR-Ub₇₅-propargylamide TIVIR-Ub-Prg to react with these DUBs was analyzed (FIG. 2B). Mutants, where the active site cysteine residue was mutated to serine failed to react.

Since the covalent linkage between Ub-Prg and DUBs was acid labile, it was postulated that this provides the perfect precipitation reagent: a chemically inert probe Ub-Prg that can be covalently immobilized onto a resin to allow for covalent target capture, stringent washing and finally acid-mediated target release. To test this, probe Ub-Prg was first immobilized covalently on CNBr-activated sepharose and various amounts of the resulting resin were incubated with a mixture of UCH-L3 and the free cysteine-containing protein bovine serum albumin (BSA), known to interfere with many proteomics investigations by its strong interactions with both resin and proteins in general. It was found that UCH-L3 could be selectively bound to the resin in the presence of BSA and, after washing with denaturing buffer and water, followed by elution with 10% trifluoroacetic acid in water, UCH-L3 could be recovered cleanly from the resin (FIG. 3). This procedure denatured UCH-L3 which could be resolubilized in chaotropic buffer for SDS-PAGE analysis.

This technology was then applied to the retrieval and identification of DUBs from cell lysate. As an initial test the well-characterized lysate of the mouse T-cell lymphoma cell line EL-4 was used, which is known to possess high DUB activity. Sepharose-bound Ub-Prg was incubated with post-nuclear EL-4 lysate for Ub-Prg hour and non-covalently bound protein was washed away using highly denaturing washing conditions. After removal of the buffer, which could interfere with subsequent mass spectrometric analysis of the retrieved DUBs, by washing with distilled water the covalently bound DUBs could be eluted using TFA in water.

Lyophilization of the resin (to remove traces of TFA) followed by resolubilizing in choatropic buffer resulted in subsequently released of purified DUBS (FIG. 3). Mass spectrometric analysis after SDS-PAGE of these isolated DUBs, showed that in a first proteomics experiment 22 members of all four families of cysteine-DUBs (USPs, UCHs, Josephins and OTU-like) could be identified.

Previous reports have suggested that thiovinyl ether intermediate can be trapped by the radical addition of a second thiol. To test this, the purified Ub-Prg -UCH-L3 complex was incubated with ethane thiol (50 mM) and radical initiator VA-044 (10 mM) for three hours. LC-MS analysis did not show the formation of an EtSH-adduct of the complex. Instead, and to our surprise, cleavage was observed of the complex with a mass corresponding to Ub-Prg plus two equivalents of ethane thiol while a mass corresponding to unmodified UCH-L3 was also observed. This cleavage reaction was confirmed by SDS-PAGE analysis.

### Conclusion

In conclusion, it has been shown that a terminal alkyne function on ubiquitin can lead to an unexpected and very efficient reaction of the active site cysteine nucleophile present in DUBs with the alkyne moiety. This finding is rather unexpected as alkynes, widely used in “click reaction” procedures are considered fully inert. These alkyne-based probes are conveniently prepared, they can be either directly immobilized onto resins for activity-based DUB proteomics or they can be conveniently fluorescently labeled for activity profiling applications with great sensitivity. In addition it has been shown that DUBs that are captured in this manner can be released in an active state by a thioradical-mediated cleavage procedure. Although here total chemical synthesis was used, the findings described here can be readily translated to a wide range of proteins through established intein-based procedures.

Experiment 2: Labeling of SENPs with SUMO-PRG Peptides

SUMO attachment to its target is similar to that of ubiquitin (as it is for the other ubiquitin-like proteins such as NEDD 8). A C-terminal peptide is cleaved from SUMO by a protease. In human these are the SENP proteases.

The following experiment was performed to confirm that propargyl modified SUMO peptide (fragments) in accordance with the invention are selective capturing agent for the corresponding SENPs. The following SUMO peptides and SENPs were used in the Experiment.
SUMO peptides:
SUMO1=Cy5-PEG4-YQEQTG-PRG
SUMO2,3=Cy5-PEG4-FQQQTG-PRG
SENPs:
GST-SENP1 (43 kDa)
SENP6 (33.1 kDa)
SENP7 (37.2 kDa)

The SENPs (1 μM final concentration) were incubated with the SUMO-PRG peptides (50 μM final concentrations) for 1 hour at RT in buffer (20 mM Tris, 100 mM NaCl, 1 mM DTT, pH 7.5). Samples were denatured using 1× LB and boiled for 5 min. As a control, the SENPs were denatured (1×LB and 5 min. boiling) prior to treatment with the SUMO-PRG peptides. Samples were resolved by SDS-PAGE (12% polyacryl amide, MES buffer) and proteins were visualized by fluorescence (λ_{ex,em}=625,680 nm) and CBB staining. The result is displayed in FIG. 4.

The selective capturing of SENP by propargyl modified SUMO peptides could be confirmed on the basis of these results.

### Experiment 3: Synthesis of Caspase-1 Probe

The following experiment was performed to confirm that the invention is generally applicable to other types of cysteine protease. For this purpose (C-terminal) propargyl modified fragments of Il-1 β, a natural substrate of caspase-1, were prepared, following the procedure schematically depicted in FIG. 5, and the capability of these probes to selectively capture caspase-1 was evaluated.

### (S)-Tent-Butyl 3-((Tert-Butoxycarbonyl)Amino)-4-Hydroxybutanoate (2)

Boc-L-Asp(tBu)-OH DCHA salt (18.8 g, 40.0 mmol) was dissolved in THF (40 mL) and cooled to −10° C. N-methylmorpholine (4.62 mL, 42.0 mmol) was added and the reaction was stirred for 5 minutes. Isobutyl chloroformate (5.45 mL, 42.0 mmol) was added drop wise over a period of 10 minutes and the reaction was stirred for another 30 minutes. Next, the solids were removed by filtration over a pad of Celite and the filtrate was collected in a cooled (0° C.) solution of NaBH₄ (3.0 g, 70.0 mmol) in water (80 mL). The reaction was stirred for 1 hour at 0° C. and then allowed to warm to room temperature. The solution was diluted with 80 mL EtOAc and extracted with 1 M HCl, water, saturated NaHCO₃ and brine. The organic layer was dried over MgSO₄ and concentrated under reduced pressure. The title compound was obtained after purification by column chromatography (20% →33% EtOAc/hexane) as a colorless oil (yield: 4.7 g, 17.0 mmol, 42%). The spectroscopic data corresponded with those reported in literature (Erwing W. R. et al. J. Med. Chem.; 1999; 42; 18; 3557-3571).

### (S)-Tent-Butyl 3-((Tert-Butoxycarbonyl)Amino)-4-Oxobutanoate (3)

A solution of oxalyl chloride (2.5 mL, 29.0 mmol) in DCM (42 mL) was cooled to -70 ° C. and to this was added a solution of DMSO (4.8 mL, 68.0 mmol) in DCM (10 mL) drop wise over a period of 30 minutes. Next, a solution of alcohol intermediate 2 (4.7 g, 17.0 mmol) in DCM (20 mL) was added drop wise over 30 minutes and the reaction was stirred for another 15 minutes at −70° C. Finally, triehtylamine (15.4 mL, 110.5 mmol) in DCM (42 mL) was added drop wise in 20 minutes after which the reaction was stirred for 1 hour at −70° C. The reaction mixture was diluted with 400 mL Et₂O and extracted three times with an aqueous 0.5 M KHSO₄ solution. It was concentrated to approximately half its volume and diluted with another 200 mL Et₂O, followed by extraction with water and brine. The organic layer was dried over MgSO₄ and concentrated under reduced pressure. The crude aldehyde was subjected to the next step without purification.

### (S)-Tent-Butyl 3-((Tert-Butoxycarbonyl)Amino)Pent-4-Ynoate (4)

This compound was synthesized using a reported procedure (Roth, G. J.; Liepold, B.; Muller, S. G.; Bestmann, H. J. Synthesis; 2004; 1; 59-62). Dimethyl-2-oxopropyl-phosphonate (2.76 mL, 20.5 mmol) was added to a suspension of K₂CO₃ (7.0 g, 51.0 mmol) and p-toluenesulfonylazide (4.0 g, 20.5 mmol) in ACN (255 mL) and the mixture was stirred vigorously for 2 hours. Crude aldehyde 3 (˜17.0 mmol) was dissolved in MeOH (50 mL) and this solution was added to the first reaction mixture. Stirring was continued for 14 hours after which the solvents were evaporated under reduced pressure. Residual solids were dissolved in Et₂O (150 mL) and water (150 mL) and the layers were separated. The organic layer was washed with water and brine, dried over MgSO₄ and concentrated under reduced pressure. The title compound was obtained after purification by column chromatography (DCM isocratic) as a colorless oil (yield: 1.89 g, 7.0 mmol, 41%). ¹H NMR (300 MHz, CDCl₃) δ 5.32 (bs, 1 H), 4.74-4.71 (m, 1 H), 2.68-2.53 (m, 2 H) 2.27 (d, J=2.4 Hz, 1 H), 1.46 (s, 9 H), 1.44 (s, 9 H). ¹³C NMR (75 MHz, CDCl₃) δ 169.42, 154.59, 82.17, 81.54, 71.02, 41.24, 39.51, 28.31, 28.04.

### (S)-Tent-Butyl 3-Aminopent-4-Ynoate Hydrochloride (5)

This compound was prepared using a reported procedure for the removal of a Boc group in the presence of a tent-butyl ester (Han, G.; Tamaki, M.; Hruby, V. J. J. Peptide Res.; 2001; 58; 338-341). A solution of HCl in dioxane (4 M, 72 mL) was cooled to 0° C. under argon and this was added to a cooled (0° C.) flask containing compound 4 (930 mg, 3.5 mmol). The ice bath was removed and the mixture was stirred for 30 minutes after which the mixture was concentrated under reduced pressure at room temperature. The residue was washed with dry Et₂O and the title compound was collected by filtration (yield: 780 mg, 2.3 mmol, 65%) as a colorless solid. ¹H NMR (300 MHz, MeOD) δ 4.42 (td, J=6.7, 2.4 Hz, 1 H), 3.28 (d, J=2.4 Hz, 1 H), 2.84 (d, J=6.7 Hz, 2 H), 1.50 (s, 9 H). ¹³C NMR (75 MHz, MeOD) δ 169.91, 83.86, 78.67, 78.19, 40.71, 39.70, 28.40.

Labeling of Recombinant Caspase-1 with Cy5-IL-1 β-Prg Probes

Caspase-1 (100 U/μL, 15 μL) was diluted in phosphate buffer (100 mM, pH 7.5) to 50 DTT (0.5 M, 0.5 μL) was added and the enzyme was incubated at room temperature for 5 minutes. 20 μL of the enzyme solution was placed in a separate vessel and iodoacetamide (200 mM, 2 μL) was added. This reaction was incubated in the dark for 10 minutes, whilst the non-IAc treated caspase-1 was incubated on ice for this duration. To 10 μL of both IAc and non-IAc treated caspase-1, 5 μL of either Cy5-IL-1 β-(93-116)-Prg or Cy5-IL-1 β-(82-116)-Prg (0.1 mg/ml) was added. To one 10 μL portion of enzyme solution 5 μL of buffer were added as a control. The mixtures were incubated at 37° C. for 3 hours prior to the addition of 4× LDS sample buffer containing 300 mM DTT. The samples were then analysed by SDS-page. After SDS-PAGE, probe reactivity was analysed on the ProExpress fluorescence scanner (625/680 nm) to detect CY-5 label. The gels were then transferred onto PVDF-membrane and analysed by Western blot using a polyclonal rabbit anti-caspase antibody (Enzo Life Science; ALX-210-804, 1:1000 dilution in 5% milk powder in PBS+ 0.1% Tween20) and a secondary goat-anti-rabbit HRP conjugate.

Labeling of Recominant Caspase-1 in Cell Lysates with Cy5-IL-1 β-Prg Probes

1*10⁹ U937 cells were lysed in PBS (100 mL) by sonicating for 10 times 20 seconds on ice. The insoluble fraction was removed by centrifugation (4000 g, 45 minutes). Protein concentration of the supernatant was 7 mg/mL as determined by NanoDrop. To 20 μL of this lysate, recombinant caspase-1 (100 U/μL; 7 μL) was added (or a buffer control) and DTT (50 mM; 3 μL). The mixtures were then incubated on ice for 10 minutes. An aliquot (10 μL) of both the caspase-1 containing and untreated lysate were then removed and iodoacetamide was added to these portions (200 mM, 1 μL). The lysates were then all incubated at room temperature in the dark for 15 minutes. To the IAc-treated samples and to one aliquot of the non-IAc treated samples (10 μL), Cy5-IL-1β-(93-116)-Prg (1 mg/ml, 1 μl) was added. The mixtures were then incubated at 37° C. for 2 hours and analysed by SDS-page. After SDS-PAGE, probe reactivity was analysed on the ProExpress fluorescence scanner (625/680 nm) to detect CY-5 label.

The selective capturing of caspase-1 by the (C-terminal) propargyl modified fragments of IL-1 β could be confirmed on the basis of these results.

### DESCRIPTION OF THE FIGURES

FIG. 1: A. Substrate turnover assay performed using UCH-L3; a decrease in polarization (mP) indicates cleavage of the isopeptide bond between G76 and Lys-Gly-TMR conjugate (15) B. (top) Deconvoluted mass of UCH-L3 (Mw_{avg}: 26181 Da). (bottom) UCH-L3 after reaction with UB-Prg results in an increase of the mass by 8544 Da corresponds to exactly Mw_{avg} of 1. C. SDS-PAGE analysis of the reaction between UB-Prg and UCH-L3.

FIG. 2: A. In vitro labeling reaction of cysteine proteases with 1. UCH-L3, catalytic subunit of USP7 (17) and CCHFV OTU-domain are three DUBs from different clades. Whereas SENP-6 is a ubiquitin-like isopeptidase and is thus expected not to react, as well as the Ub activating enzyme UBE1 and papain, a general cysteine protease obtained from Papaya Latex. B. Lysate labeling. C. Labeling reactions in cell lysates, analyzed by Western blot. GFP fusions of DUBs from the USP, OTU were transfected in HeLa cells and their reaction with UB-Prg indirectly shown using anti-GFP western blot. DUBs annotated with CS are catalytic cysteine to serine mutants.

FIG. 3: (Left) SDS-PAGE gel showing the selective binding of UCHL3 to sepharose beads functionalized with UB-Prg in the presence of excess bovine serum albumin. Control resin does not bind UCHL3. (Right) Washing of UCHL3 functionalized beads, with subsequent TFA mediated release and precipitation. Final resolubilization shows recovery of UCHL3 from sepharose.

FIG. 4: SDS-PAGE gel showing the selective binding of propargyl modified SUMO peptides to SENPs. Incubation of SUMO-hydrolases SENP1, 6 and 7 with truncated fluorescent C-terminal propargylated SUMO peptides. ‘SUMO1-peptide’ corresponds to the C-terminus of SUMO-1, and ‘SUMO2-peptide’ and ‘SUMO3-peptide’ correspond to the C-termini of SUMO-2 and SUMO-3 respectively. Denaturing of the hydrolases prior to addition of the peptides completely abolishes binding.

FIG. 5: Synthesis of caspase-1 probe: Prg-Asp was synthesized as shown and attached to the C-terminus of peptide spanning two fragments of IL-10, the natural substrate of caspase-1

FIG. 6: Labeling of (A) recombinant caspase-1 with the two different caspase-1-probes. Both labeled the lysate with equal affinity; (B) of activated caspase-1 doped into cell lysate. No background labeling is observed.

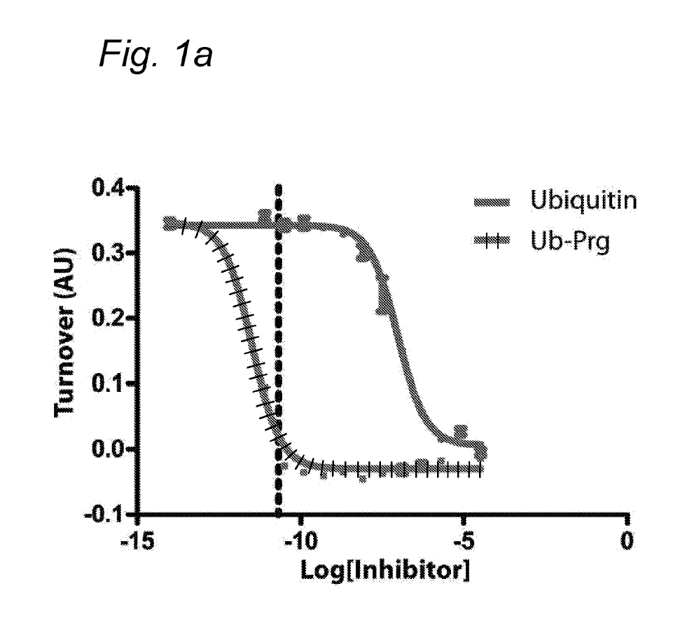

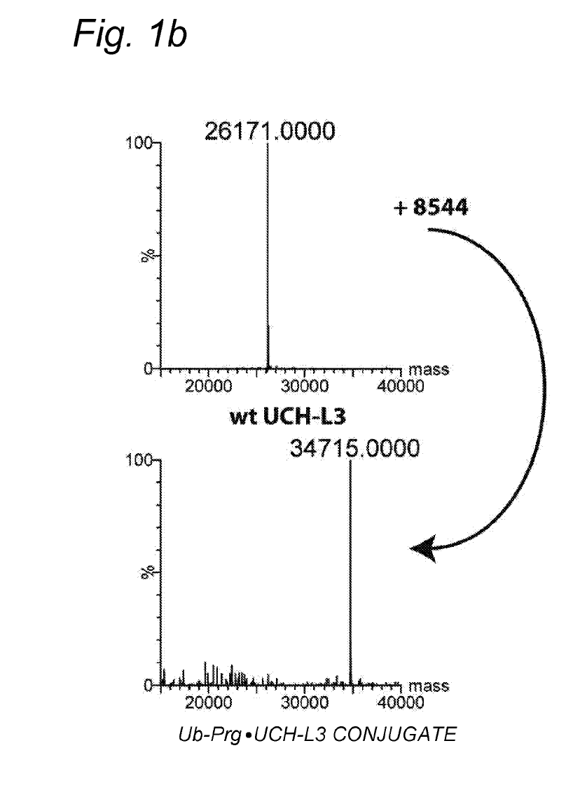

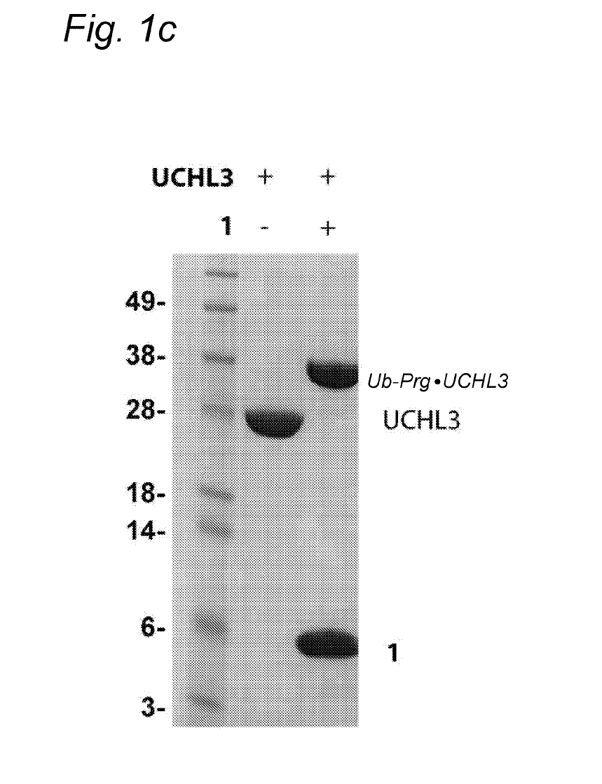

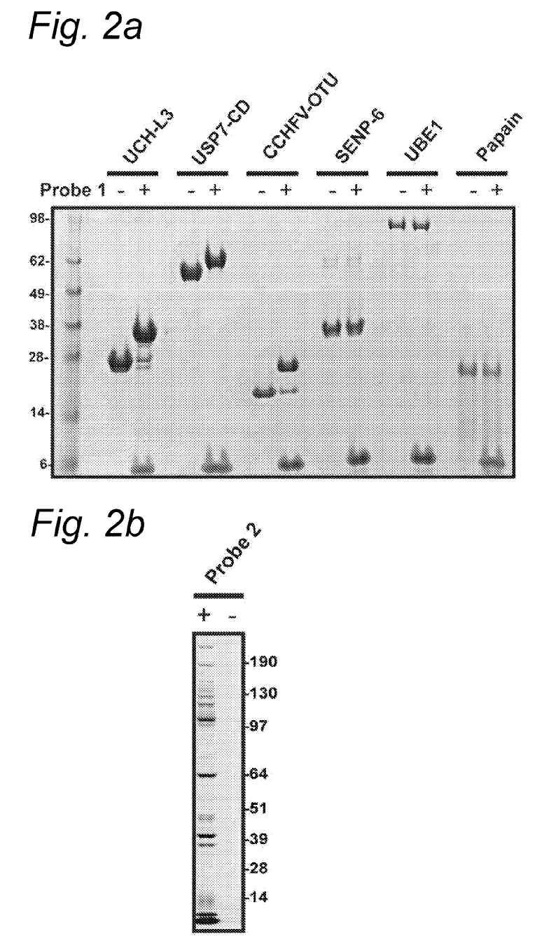

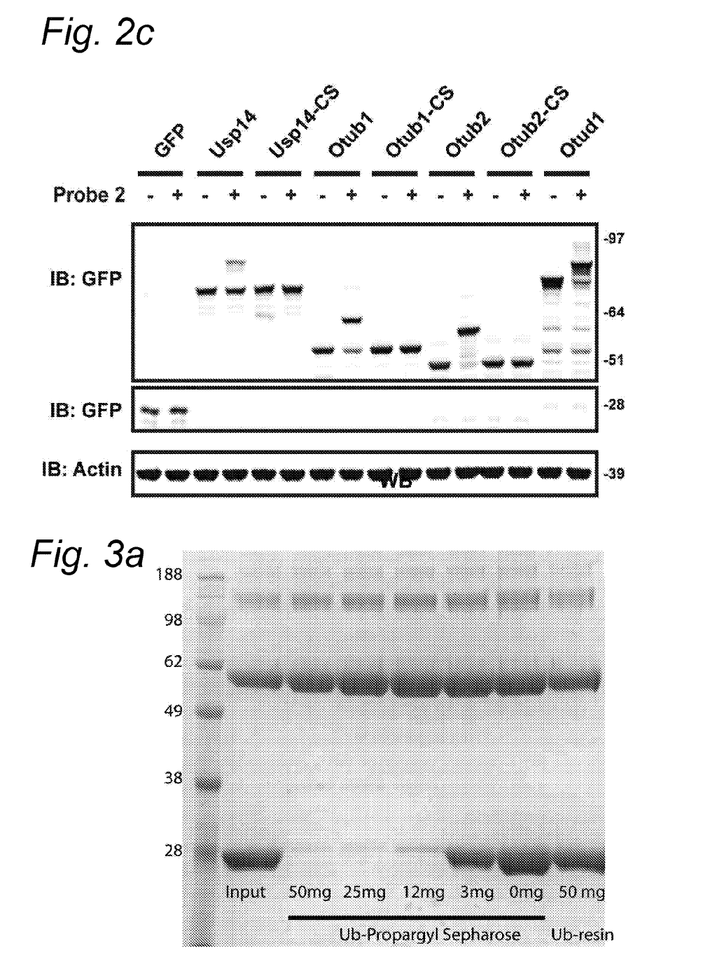

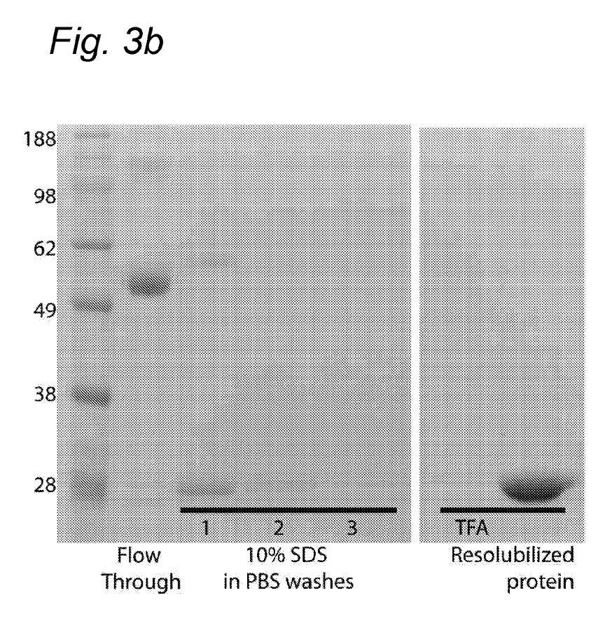

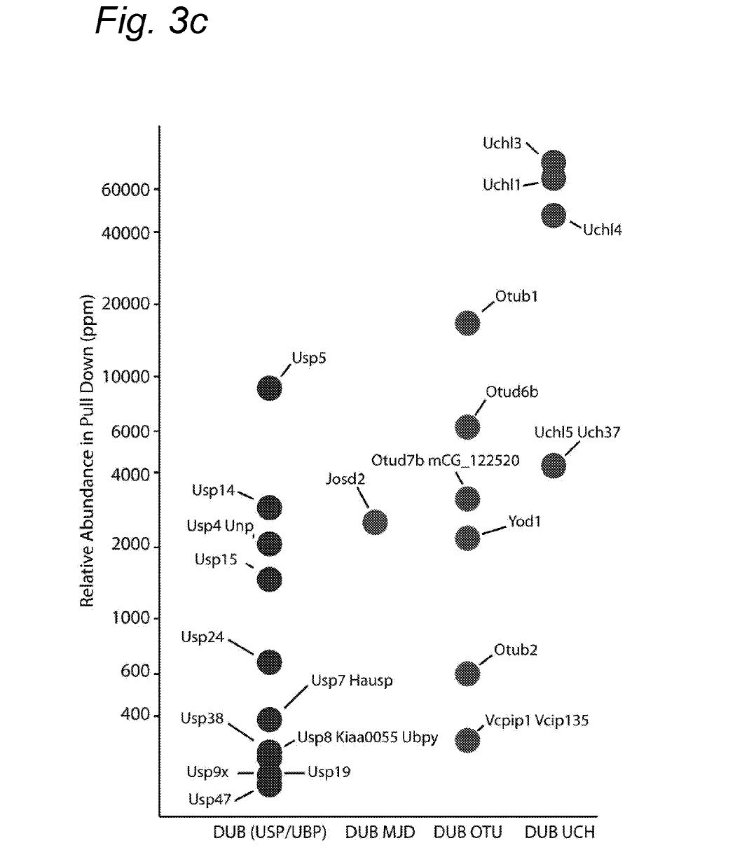

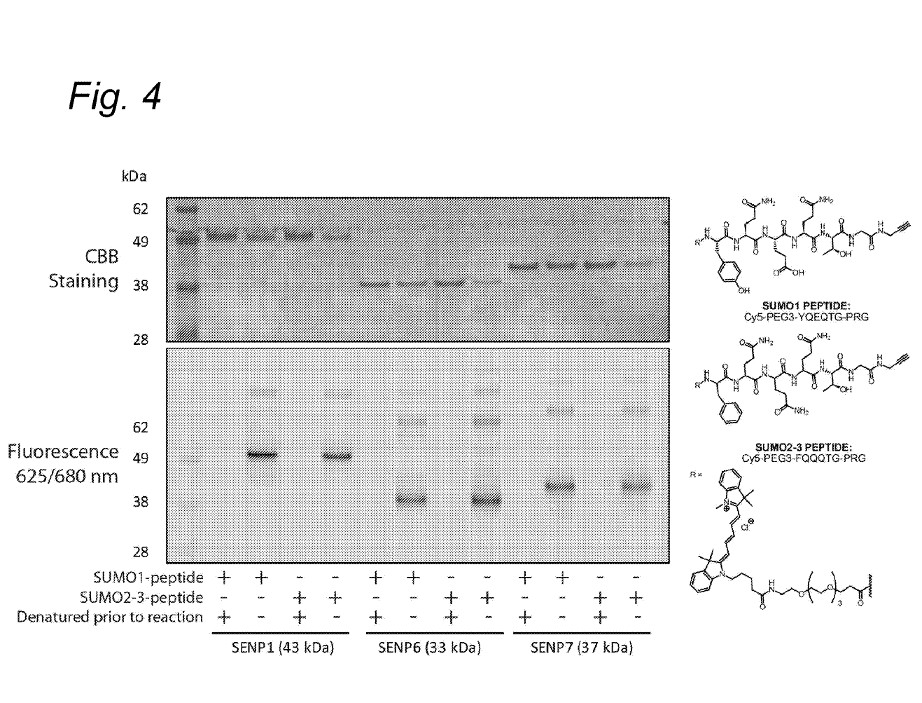

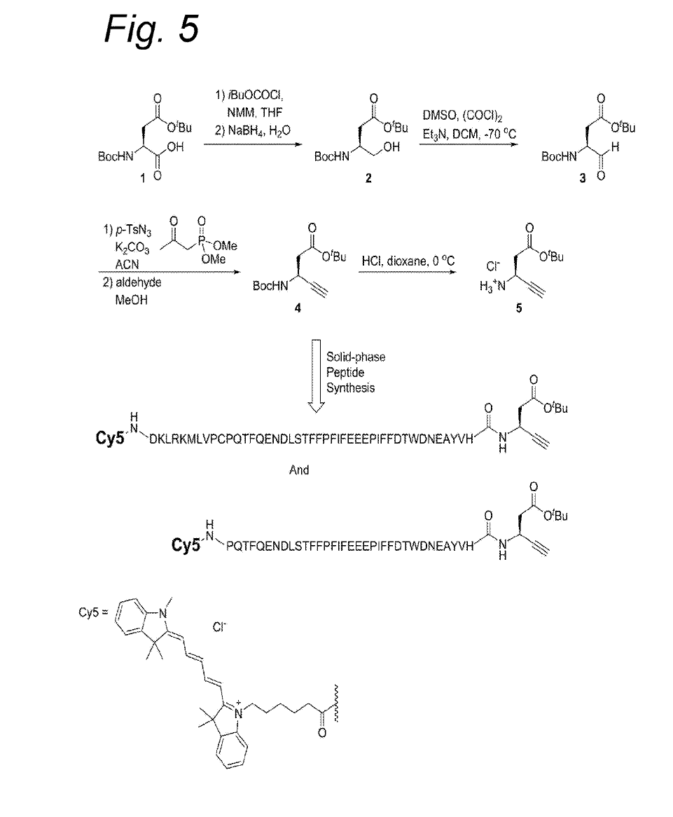

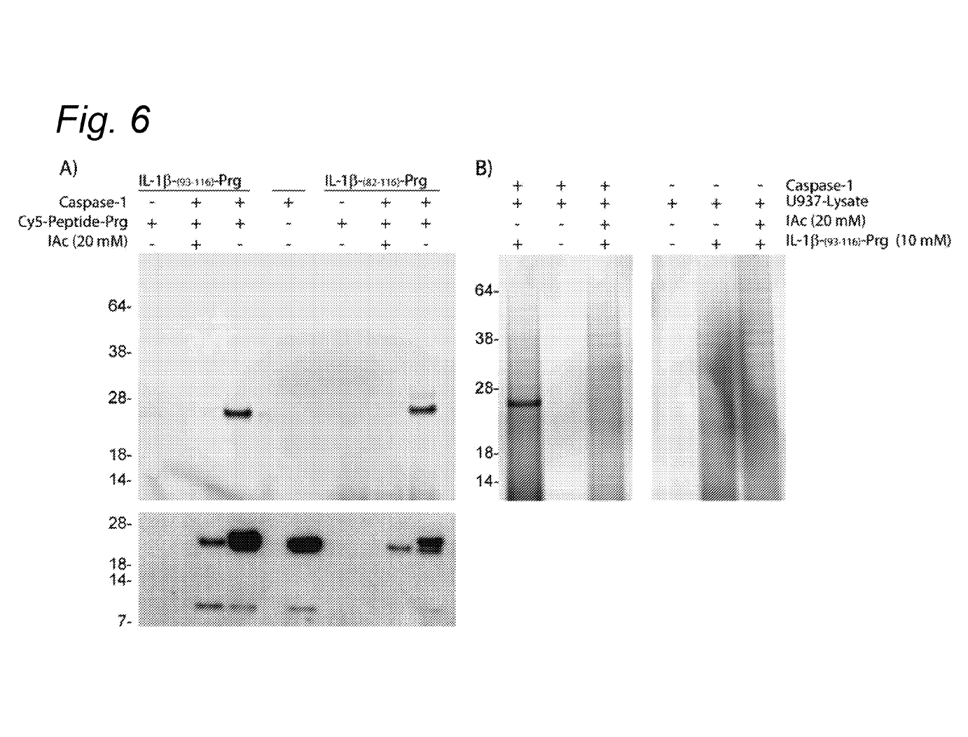

TABLE 1
natural and non-natural cysteine protease substrates

| | | SEQ |
|---|---|---|
| | | ID |
| Ub (mutant) | Sequence (a^{-p}→⁻¹) | NO. |
| Ub | MQIFVKTLTGKTITLEVEPSDTIENVKAKIQDKEGIPPDQQRLIFAGKQLEDGRTLSDYNIQKESTLHLVLRLRGG | 1 |
| UbM1C | CQIFVKTLTGKTITLEVEPSDTIENVKAKIQDKEGIPPDQQRLIFAGKQLEDGRTLSDYNIQKESTLHLVLRLRGG | 2 |
| HA-Ub | YPYDVPDYAMQIFVKTLTGKTITLEVEPSDTIENVKAKIQDKEGIPPDQQRLIFAGKQLEDGRTLSDYNIQKESTLHLVLRL | 3 |
| | RGG | |
| His6-Ub | HHHHHHMQIFVKTLTGKTITLEVEPSDTIENVKAKIQDKEGIPPDQQRLIFAGKQLEDGRTLSDYNIQKESTLHLVLRLRGG | 4 |
| (D-Arg)8-Ub | rrrrrrrrMQIFVKTLTGKTITLEVEPSDTIENVKAKIQDKEGIPPDQQRLIFAGKQLEDGRTLSDYNIQKESTLHLVLRLR | 5 |
| | GG | |
| Penetratin-Ub | RQIKWFQNRRMQIFVKTLTGKTITLEVEPSDTIENVKAKIQDKEGIPPDQQRLIFAGKQLEDGRTLSDYNIQKESTLHLVLR | 6 |
| | LRGG | |
| Tat-Ub | YGRKKRRQRRRMQIFVKTLTGKTITLEVEPSDTIENVKAKIQDKEGIPPDQQRLIFAGKQLEDGRTLSDYNIQKESTLHLVL | 7 |
| | RLRGG | |
| UbM1(OrnN2) | (OrnN2)QIFVKTLTGKTITLEVEPSDTIENVKAKIQDKEGIPPDQQRLIFAGKQLEDGRTLSDYNIQKESTLHLVLRLRGG | 8 |
| UbK6(OrnN2) | MQIFV(OrnN2)TLTGKTITLEVEPSDTIENVKAKIQDKEGIPPDQQRLIFAGKQLEDGRTLSDYNIQKESTLHLVLRLRGG | 9 |
| UbK11(OrnN2) | MQIFVKTLTG(OrnN2)TITLEVEPSDTIENVKAKIQDKEGIPPDQQRLIFAGKQLEDGRTLSDYNIQKESTLHLVLRLRGG | 10 |
| UbK27(OrnN2) | MQIFVKTLTGKTITLEVEPSDTIENV(OrnN2)AKIQDKEGIPPDQQRLIFAGKQLEDGRTLSDYNIQKESTLHLVLRLRGG | 11 |
| UbK29(OrnN2) | MQIFVKTLTGKTITLEVEPSDTIENVKA(OrnN2)IQDKEGIPPDQQRLIFAGKQLEDGRTLSDYNIQKESTLHLVLRLRGG | 12 |
| UbK33(OrnN2) | MQIFVKTLTGKTITLEVEPSDTIENVKAKIQD(OrnN2)EGIPPDQQRLIFAGKQLEDGRTLSDYNIQKESTLHLVLRLRGG | 13 |
| UbK48(OrnN2) | MQIFVKTLTGKTITLEVEPSDTIENVKAKIQDKEGIPPDQQRLIFAG(OrnN2)QLEDGRTLSDYNIQKESTLHLVLRLRGG | 14 |
| UbK63(OrnN2) | MQIFVKTLTGKTITLEVEPSDTIENVKAKIQDKEGIPPDQQRLIFAGKQLEDGRTLSDYNIQ(OrnN2)ESTLHLVLRLRGG | 15 |
| UbK6(δ-thioK) | MQIFVKTLTG( [symbol] )TITLEVEPSDTIENVKAKIQDKEGIPPDQQRLIFAGKQLEDGRTLSDYNIQKESTLHLVLRLRGG | 16 |
| UbK11(δ-thioK) | MQIFVKTLTG( [symbol] )TITLEVEPSDTIENVKAKIQDKEGIPPDQQRLIFAGKQLEDGRTLSDYNIQKESTLHLVLRLRGG | 17 |
| UbK27(δ-thioK) | MQIFVKTLTGKTITLEVEPSDTIENV( [symbol] )AKIQDKEGIPPDQQRLIFAGKQLEDGRTLSDYNIQKESTLHLVLRLRGG | 18 |
| UbK29(δ-thioK) | MQIFVKTLTGKTITLEVEPSDTIENVKA( [symbol] )IQDKEGIPPDQQRLIFAGKQLEDGRTLSDYNIQKESTLHLVLRLRGG | 19 |
| UbK33(δ-thioK) | MQIFVKTLTGKTITLEVEPSDTIENVKAKIQD( [symbol] )EGIPPDQQRLIFAGKQLEDGRTLSDYNIQKESTLHLVLRLRGG | 20 |
| UbK48(δ-thioK) | MQIFVKTLTGKTITLEVEPSDTIENVKAKIQDKEGIPPDQQRLIFAG( [symbol] )QLEDGRTLSDYNIQKESTLHLVLRLRGG | 21 |
| UbK63(δ-thioK) | MQIFVKTLTGKTITLEVEPSDTIENVKAKIQDKEGIPPDQQRLIFAGKQLEDGRTLSDYNIQ( [symbol] )ESTLHLVLRLRGG | 22 |
| UbK48(γ-thioK) | MQIFVKTLTGKTITLEVEPSDTIENVKAKIQDKEGIPPDQQRLIFAG( [symbol] )QLEDGRTLSDYNIQKESTLHLVLRLRGG | 23 |
| UbL43photoLeu | MQIFVKTLTGKTITLEVEPSDTIENVKAKIQDKEGIPPDQQR(photoLeu)IFAGKQLEDGRTLSDYNIQKESTLHLVLRLR | 24 |
| | GG | |
| UbL71photoLeu | MQIFVKTLTGKTITLEVEPSDTIENVKAKIQDKEGIPPDQQRLIFAGKQLEDGRTLSDYNIQKESTLHLV(photoLeu) | 25 |
| | RLRGG | |
| UbL73photoLeu | MQIFVKTLTGKTITLEVEPSDTIENVKAKIQDKEGIPPDQQRLIFAGKQLEDGRTLSDYNIQKESTLHLVLR(photoLeu) | 26 |

RGG

1-18. (canceled)

19. A cysteine protease capturing agent, comprising a modified C-terminal portion of a C→N cleavage fragment of a cysteine protease substrate, wherein the cysteine protease capturing agent is represented by formula (I): 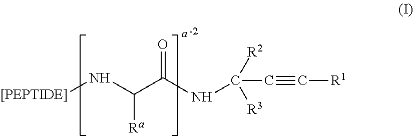
wherein:
R¹ represents hydrogen or a substituent selected from —F, —CF₃, —CHF₂, —CH₂F, —Cl, —CCl₃, —CHCl₂ and —CH₂Cl;
R^{a} represents an amino acid side chain identical to the amino acid side chain of the corresponding amino acid of the cysteine protease substrate;
R² and R³are independently selected from the group consisting of hydrogen, —F, —CF₃, —CHF₂, —CH₂F, —Cl, —CCl₃, —CHCl₂ and —CH₂Cl or one of R² and R³ represents a natural amino acid side chain, while the other represents hydrogen; and
[PEPTIDE] represents a peptide chain comprising an amino acid sequence corresponding to a^{-p}-a⁻³; or an N-terminally truncated variant thereof having a length of at least 2 amino acid residues; or a homologue or conjugate thereof; wherein a^{#} indicates the amino acid residue position in the corresponding intact cysteine protease substrate relative to the cleavage site thereof, a¹ and a⁻¹ being defined as the amino acid residues adjacent to the cleavage site; and
wherein p represents an integer equal to the total number of amino acids of the C→N cleavage fragment of the cysteine protease substrate.

20. The cysteine protease capturing agent according to claim 19, wherein one of R² and R³ represents the amino acid side chain of a⁻¹, while the other represents hydrogen.

21. The cysteine protease capturing agent according to claim 19, wherein the cysteine protease capturing agent is not Ub74-propargylamide, Ub75-propargylamide, Ub76-propargylamide, alkyne-Leu-Leu-NH₂, alkyne-Leu-Leu-Phe-Leu-Val-N₃, Ac-Tyr-Gly-Gly-Phe-Leu-Prop, Ac-Tyr-Gly-Pgl-Phe-Leu-NH₂, Boc-protected or unprotected Lys-Lys(Lys)-Prop or Boc protected or unprotected Lys-Lys(Lys)-Lys(Lys(Lys)-Lys)-Prop.

22. The cysteine protease capturing agent according to claim 19, wherein R¹ represents hydrogen.

23. The cysteine protease capturing agent according to claim 19, wherein [PEPTIDE] represents an amino acid sequence having a length of at least 3.

24. The cysteine protease capturing agent according to claim 19, wherein the cysteine protease is selected from the group of consisting of deubiquitinating proteases, cathepsins, calpains, caspases and SUMO proteases, preferably from the group consisting of deubiquitinating proteases, SUMO protease, caspases and cathepsins.

25. The cysteine protease capturing agent according to claim 19, wherein a^{-p}-a⁻¹ represents an amino acid sequence selected from the group consisting of SEQ ID no. 1-35.

26. The cysteine protease capturing agent derivative according to claim 19, further comprising a ligand selected from the group of fluorophores, affinity labels, biophysical labels, chelating agents, complexing agents and epitope tags.

27. A method of diagnosing, treating or preventing a disease or disorder involving the action of a cysteine protease, comprising administering to a patient in need thereof a cysteine protease capturing agent according to claim 19.

28. The method according to claim 27, wherein the disease or disorder is an auto immune disease, cancer, infection or lysosomal storage disease.

29. A method of inhibiting cysteine protease activity, comprising exposing the cysteine protease to a cysteine protease capturing agent according to claim 19.

30. A cysteine protease capturing agent comprising a modified N-terminal portion of the N→C fragment of a cysteine protease substrate, wherein the cysteine protease capturing agent is represented by formula (II): 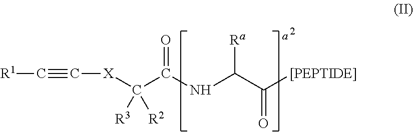
wherein:
R¹ represents hydrogen or a substituent selected from —F, —CF₃, —CHF₂, —CH₂F, —Cl, —CCl₃, —CHCl₂ and 13 CH₂Cl;
R^{a} represents an amino acid side chain identical to the amino acid side chain of the corresponding amino acid of the cysteine protease substrate;
R² and R³are independently selected from the group of hydrogen, —F, —CF₃, —CHF₂, —CH₂F, —Cl, —CCl₃, —CHCl₂ and —CH₂Cl or one of R² and R³ represents a natural amino acid side chain, while the other represents hydrogen;
—X— represents a covalent bond or a moiety selected from —NH— and —CR⁴R⁵—, wherein R⁴ and R⁵ are independently selected from the group consisting of hydrogen, —F, —CF₃, —CHF₂, —CH₂F, —Cl, —CCl₃, —CHCl₂ and —CH₂Cl; and
[PEPTIDE] represents a peptide chain having an amino acid sequence corresponding to a³-a^{q}; or a C-terminally truncated variant thereof having a length of at least 2 amino acid residues; or a homologue or conjugate thereof; wherein a^{#} indicates the amino acid residue position in the corresponding intact cysteine protease substrate relative to the cleavage site thereof, a¹ and a⁻¹ being defined as the amino acid residues adjacent to the cleavage site; and wherein q represents an integer equal to the total number of amino acids of the N→C cleavage fragment of the cysteine protease substrate.

31. The cysteine protease capturing agent according to claim 30, wherein —X— represents —NH—.

32. The cysteine protease capturing agent according to claim 30, wherein the cysteine protease capturing agent is not Ub74-propargylamide, Ub75-propargylamide, Ub76-propargylamide, alkyne-Leu-Leu-NH₂, alkyne-Leu-Leu-Phe-Leu-Val-N₃, Ac-Tyr-Gly-Gly-Phe-Leu-Prop, Ac-Tyr-Gly-Pgl-Phe-Leu-NH₂, Boc-protected or unprotected Lys-Lys(Lys)-Prop or Boc protected or unprotected Lys-Lys(Lys)-Lys(Lys(Lys)-Lys)-Prop.

33. The cysteine protease capturing agent according to claim 30, wherein the cysteine protease is selected from the group of consisting of deubiquitinating proteases, cathepsins, calpains, caspases and SUMO proteases, preferably from the group consisting of deubiquitinating proteases, SUMO protease, caspases and cathepsins.

34. The cysteine protease capturing agent derivative according to claim 30, further comprising a ligand selected from the group of fluorophores, affinity labels, biophysical labels, chelating agents, complexing agents and epitope tags.

35. A method of diagnosing, treating or preventing a disease or disorder involving the action of a cysteine protease, comprising administering to a patient in need thereof a cysteine protease capturing agent according to claim 30.

36. A cysteine protease capturing agent in the form of a peptide mimetic comprising a spatial arrangement of reactive chemical moieties and/or functional groups that resembles the three-dimensional arrangement of active and/or functional groups of a cysteine protease capturing agent according to claim 19, wherein the peptide mimetic comprises the propargyl or modified propargyl moiety of any one of the peptides and wherein the peptide mimetic is capable of being recognized by and interacting with the active site of the cysteine protease.

37. A method of purifying/isolating a cysteine protease from a biological sample, comprising:
(a) combining a sample comprising a cysteine protease with a corresponding cysteine protease capturing agent according to claim 19, wherein the cysteine protease capturing agent is conjugated to a chelating agent, a complexing agent, an epitope tag or a solid phase, which allows for or results in immobilization of the cysteine protease capturing agent; and
(b) selectively binding the cysteine protease to the cysteine protease capturing agent;
(c) separating the sample from the immobilized cysteine protease capturing agent.

38. A method of producing a selective cysteine protease binding agent, comprising introducing to a cysteine protease substrate a terminal alkyne group capable of interacting with the thiol side chain of the cysteine residue present in the active site of the cysteine protease.
